(19) United States
(12) Patent Application Publication
(10) Pub. No.: US 2014/0303658 A1
(43) Pub. Date: Oct. 9, 2014

(54) Thrombectomy Catheter System

(71) Applicant: Bayer Medical Care Inc., Indianola, PA (US)

(72) Inventors: Michael J. Bonnette, Minneapolis, MN (US); David B. Morris, Anoka, MN (US)

(21) Appl. No.: 14/310,151

(22) Filed: Jun. 20, 2014

Related U.S. Application Data
(63) Continuation-in-part of application No. 13/766,367, filed on Feb. 13, 2013.

(51) Int. Cl. A61B 17/3203

(52) U.S. Cl. CPC A61B 17/32037; A61B 2017/320052
USPC 606/159

(57) ABSTRACT

A thrombectomy catheter includes a catheter body extending from a catheter proximal portion to a catheter distal portion and including a catheter intermediate portion, the catheter body includes an aspiration lumen and an infusion lumen extending along the catheter body, wherein the aspiration lumen includes an aspiration orifice open at a distal end of the catheter body.

### FIELD

Medical devices, and more specifically to thrombectomy catheters and procedures.

### BACKGROUND

A thrombectomy is a medical procedure used to remove a blood clot (thrombus) from a vessel, such as an artery or vein. If a thrombus is not removed, it may obstruct blood flow. One technique to perform a thrombectomy is to use a catheter having an infusion lumen, used to break up the thrombus, and an aspiration lumen, used to vacuum up the thrombus and emboli.

In some examples, thrombectomy procedures are conducted with complex catheter systems configured to provide multiple jets of high pressure fluid, such as saline supplied at pressures of 10,000 psi or more. Supplying high pressure fluid correspondingly requires a high pressure pump. Pumps for a high pressure thrombectomy procedure may have limited utility for other medical procedures (e.g., medication and contrast infusion and the like).

Additionally, the thrombectomy catheters used in these procedures are constructed with complex manifolds, fluid jet exhaust features and the like to distribute jets of fluid for the removal of thrombus from a vessel. Furthermore, these catheters are constructed with robust materials to permit the delivery and distribution of high pressure fluids. These thrombectomy systems are correspondingly expensive, require multi-step manufacturing techniques and further require specialized equipment for operation (for instance a high pressure pump, as described above).

### OVERVIEW

One example of the present disclosure can include a catheter body extending from a catheter proximal portion to a catheter distal portion and including a catheter intermediate portion, the catheter body includes an aspiration lumen and an infusion lumen extending along the catheter body, wherein the aspiration lumen includes an aspiration orifice open at a distal end of the catheter body.

In another example of the present disclosure, the catheter body includes an integral homogenous cross-section profile and includes a multi-durometer hardness varying along the catheter body's length such that the catheter proximal portion has a relatively high durometer and the catheter distal portion has a relatively low durometer, with respect to each other.

In still another example of the present disclosure, the infusion lumen extends along the catheter body towards the distal portion and includes a single infusion orifice that is configured to direct a fluid jet radially away from a longitudinal axis of the catheter body.

In yet another example of the present disclosure, the distal end of the catheter body includes an aspiration orifice distal member including a proximal portion extending from the distal end of the catheter body having an opening sized similar to the aspiration lumen and a distal portion having an opening wider than the aspiration lumen.

A particular example discloses a thrombectomy catheter comprising a catheter body extending from a catheter proximal portion to a catheter distal portion; an aspiration lumen extending through the catheter body from the catheter proximal portion toward the catheter distal portion, the aspiration lumen including an aspiration orifice near the catheter distal portion, wherein the distal end of the catheter body includes an aspiration orifice distal member including a proximal portion extending from the distal end of the catheter body having an opening sized similar to the aspiration lumen and a distal portion having an opening wider than the aspiration lumen; and an infusion lumen extending along the catheter body towards the distal portion and having a single infusion orifice located in a side wall of the catheter body that is configured to direct a fluid jet radially away from a longitudinal axis of the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen includes an aspiration orifice open at a distal end of the catheter body; and wherein the infusion lumen extends along the catheter body towards the distal portion and includes a single infusion orifice that is configured to direct a fluid jet radially away from a longitudinal axis of the catheter body.

Another particular example discloses a thrombectomy catheter comprising a catheter body extending from a catheter proximal portion to a catheter distal portion and including a catheter intermediate portion, wherein the catheter proximal portion has a relatively high durometer value and the catheter distal portion has a relatively low durometer value, with respect to each other; the catheter body including an aspiration lumen extending through the catheter body from the catheter proximal portion toward the catheter distal portion, the aspiration lumen including an aspiration orifice open at a distal end of the catheter body, wherein the aspirating orifice is free from structural obstructions at the distal end of the catheter body and wherein the distal end of the catheter body includes an aspiration orifice distal member including a proximal portion extending from the distal end of the catheter body having an opening sized similar to the aspiration lumen and a distal portion having an opening wider than the aspiration lumen; the catheter body further including an infusion lumen extending along the catheter body towards the distal portion with an infusion orifice extending through the catheter body to direct a fluid jet away from the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen extends through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body a distal portion, wherein the distal portion has a greater cross-sectional area than the cross-sectional area of the proximal portion; and wherein the infusion lumen extends along the catheter body towards the distal portion with an infusion orifice extending through the catheter body to direct a fluid jet away from the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen extends through the catheter body from the catheter proximal portion toward the catheter distal portion, the aspiration lumen including an aspiration orifice open at a distal end of the catheter body; and wherein the infusion lumen extends along the catheter body towards the distal portion with an infusion orifice extending through the catheter body to direct a fluid jet away from the catheter body.

Another particular example discloses a thrombectomy catheter comprising a catheter body including an aspiration lumen extending though the catheter body and open at an aspiration orifice; an infusion body including a fluid delivery lumen extending to an infusion orifice, the infusion body extending through the aspiration lumen; and an expanded member coupled to a distal end of the infusion body and located distally from the infusion orifice.

Another particular example discloses a thrombectomy system comprising a fluid delivery device; an aspirator; and a thrombectomy catheter with a first port coupled to the fluid delivery device and a second port coupled to the aspirator, wherein the thrombectomy catheter includes: a catheter body extending from a catheter proximal portion to a catheter distal portion and including a catheter intermediate portion, wherein the catheter proximal portion has a relatively high durometer value and the catheter distal portion has a relatively low durometer value, with respect to other; an aspiration lumen extending through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body having an opening sized similar to the aspiration lumen and a distal portion having an opening wider than the aspiration lumen; the catheter body further including an infusion lumen extending along the catheter body towards the distal portion with an infusion orifice extending through the catheter body to direct a fluid jet away from the catheter body.

Another particular example discloses the thrombectomy system of the previous paragraph wherein the aspirator includes a vacuum source including a plurality of syringes ganged together via a stop cock style manifold.

Another particular example discloses the thrombectomy catheter of any of the previous paragraphs wherein the single infusion orifice is recessed proximally away from the aspiration orifice.

Another particular example discloses the thrombectomy catheter of any of the previous paragraphs wherein the distal end of the catheter body includes an aspiration orifice distal member including a proximal portion extending from the distal end of the catheter body having an opening sized similar to the aspiration lumen and a distal portion having an opening wider than the aspiration lumen.

Still another particular example discloses an injector system comprising a housing holding a high pressure pump, a low pressure pump, and an aspiration module; wherein a thrombectomy catheter is configured for coupling to the high pressure pump or the low pressure pump; the high pressure pump further comprising a single piston pump capable of delivering fluid at pressures of between 5000 psi to 10,000 psi; and the low pressure pump further comprising a multi-piston pump capable of delivering fluids at between 500 psi to 1500 psi.

Another particular example discloses the injector system of the previous paragraph wherein the high pressure pump and the low pressure pump are configured to operate independently of each other.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a thrombectomy catheter, in accordance with one embodiment of the present disclosure.

FIG. 2 shows a cross-section of the thrombectomy catheter of FIG. 1.

FIG. 3 shows a perspective view of the distal portion of the thrombectomy catheter of FIG. 1.

FIG. 4 shows side view of the distal portion of the thrombectomy catheter of FIG. 1.

FIG. 5 shows an end view of an aspiration orifice member.

FIG. 6A shows a side view of the thrombectomy catheter in a vessel with thrombus lodged in a wide mouth distal member with the thrombus annularly engaged by the wide mouth perimeter.

FIG. 6B shows a side view of the thrombectomy catheter of FIG. 6A with the thrombus collapsed within the wide mouth distal member and translated toward the proximal catheter end.

FIG. 6C shows a side view of the thrombectomy catheter of FIG. 6B with the thrombus collapsed into the smaller diameter portion of the catheter and translated toward the proximal catheter end.

FIG. 7 shows a cross-section of a thrombectomy catheter, in accordance with one embodiment of the present disclosure.

FIG. 8 shows a distal end of a thrombectomy catheter, in accordance with one embodiment of the present disclosure.

FIG. 9A shows a portion of a thrombectomy system, in accordance with one embodiment of the present disclosure.

FIG. 9B shows an injector system, in accordance with one embodiment of the present disclosure.

FIG. 10 shows a perspective view of a vacuum source, in accordance with one embodiment of the present disclosure.

FIG. 11 shows a front view of the vacuum source of FIG. 10.

FIG. 12A shows a schematic view of one example of an injector system, according to one embodiment of the present disclosure.

FIG. 12B shows a schematic view of one example of an injector system, according to one embodiment of the present disclosure.

FIG. 13A shows a side view of a thrombectomy catheter according to one embodiment of the present disclosure.

FIG. 13B shows a detailed cross sectional view of an expanded member for used with the thrombectomy catheter shown in FIG. 13A.

FIG. 14 shows a detailed view of the thrombectomy catheter of FIG. 13A with a plug at an aspiration orifice.

FIG. 15 shows another detailed side view of the thrombectomy catheter of FIG. 14 with the plug mechanically driven into an aspiration lumen.

FIG. 16 shows a side view of another example of a thrombectomy catheter according to one embodiment of the present disclosure.

FIG. 17 shows a detailed schematic view of the catheter distal portion of the thrombectomy catheter of FIG. 16

FIG. 18A shows a detailed view of the thrombectomy catheter of FIG. 16 in the deployed position with the aspiration orifice at least partially blocked.

FIG. 18B shows a detailed view of the thrombectomy catheter of FIG. 18A in a retracted and plunging position with the blockage in the aspiration lumen

FIG. 19 shows a schematic view of one example of a thrombectomy catheter system including a double action infusion pump.

FIG. 20 shows an exploded view of the double action infusion pump of FIG. 19.

FIG. 21 shows a perspective schematic view of the double action infusion pump of FIG. 20.

FIG. 22 shows a schematic diagram of a cylinder and piston of the double action infusion pump in two configurations.

FIG. 23 shows a schematic view of another example of a thrombectomy catheter system including a single action infusion pump.

### DETAILED DESCRIPTION

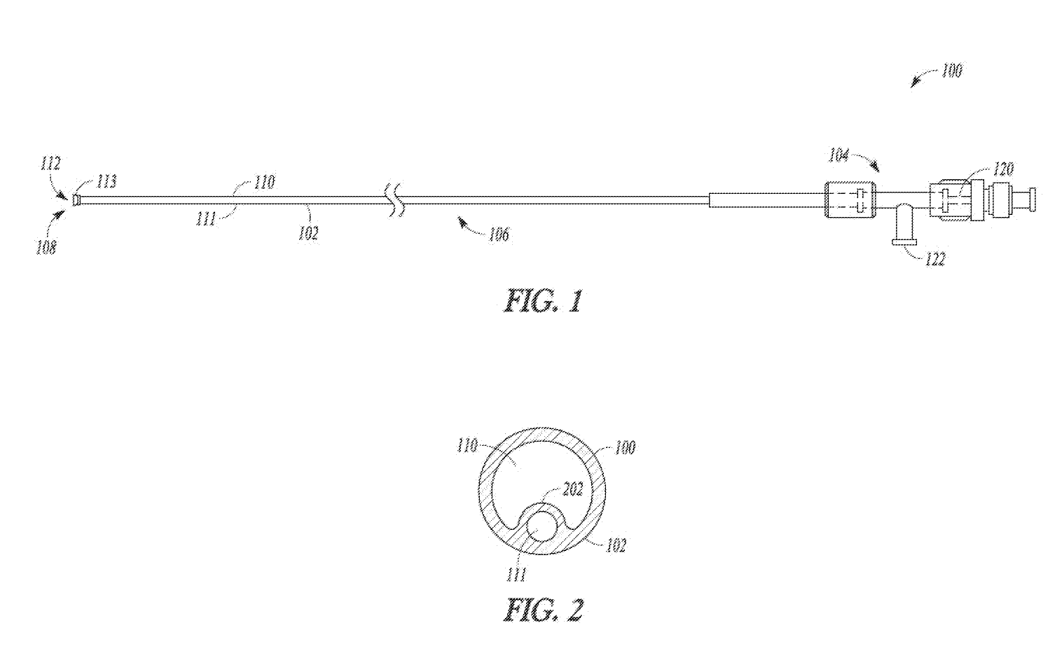

FIG. 1 shows a side view of a thrombectomy catheter 100 in accordance with one embodiment of the present disclosure. As will be described in detail below, the thrombectomy catheter 100 is configured to provide a pressurized fluid at a distal end for the removal of thrombus from a vessel. Additionally, the thrombectomy catheter is configured to provide a vacuum source (aspiration) at the catheter distal end for removal of thrombus removed with the pressurized fluid. The thrombectomy catheter 100 generally includes a catheter body 102 extending from a catheter proximal portion 104 to a catheter distal portion 108. A catheter intermediate portion 106 extends between catheter proximal and distal portions 104, 108. The catheter body 102 is configured, in one example to provide a catheter distal portion 108 more flexible than the catheter proximal portion 104 to facilitate the navigation of the catheter body 102 through vasculature of the subject. The catheter body 102 includes an aspiration lumen 110 and an infusion lumen 111 extending along the catheter body 102 from the catheter proximal portion 104 toward the catheter distal portion 108.

Referring to FIG. 1, the infusion lumen 111 is coupled to a side port 122 that can be coupled to a fluid delivery device, as will be discussed below. The infusion lumen 111 is configured to deliver fluid under pressure to the catheter distal portion 108, for example, to a jet orifice used in a thrombectomy procedure. The jet orifice provides a jet of the fluid at pressures of around 1500 psi or in a range of around 500 to 1500 psi for hydrodynamic engagement with thrombus although other pressures may be obtained. The aspiration lumen 110 is coupled to a central port 120 that can be coupled to a vacuum apparatus.

In use, the thrombectomy catheter 100 is inserted into a vessel, such as a vein or artery, and fluid is delivered to the catheter distal portion 108 via the infusion lumen 111. The fluid is delivered through one or more jets, and hydrodynamically breaks up thrombus within the vessel (e.g., through concentrated fluid pressure, fluid velocity, and fluid flow volume). For instance, the fluid impacts the thrombus and mechanically macerates the thrombus through this engagement. As discussed below, the aspiration lumen 110 receives the broken up thrombus, through a widened aspiration orifice distal member 114, and delivers it through port 120 to a waste unit such as a collection bag, vial, chute and the like.

### Catheter Body

In one embodiment, the catheter body 120 is formed such that the distal portion 108 is relatively flexible, and the proximal portion 104 is stiff relative to the distal portion 108. Relative flexibility of the distal portion 108 allows the catheter body 120 to flexibly follow or navigate the vessel for ease of insertion. The stiffer proximal portion 104 of the catheter body 120 allows for more torqueability and easier advancement along a guide wire, for example. In one example, approximately the distal 6 inches of the catheter body 120 has a lower durometer hardness than the rest of the catheter body. One exemplary catheter uses 6533 PEBAX for the distal portion 108 and 7233 PEBAX for the proximal portion 104, with the 7233 PEBAX having a lower durometer value than the 6533 PEBAX.

In another example, the catheter proximal portion 104 has a high durometer value, the catheter intermediate portion 106 has a relatively medium durometer value, and the catheter distal portion 108 has a relatively low durometer value, with respect to each of the other of the proximal, intermediate, and distal catheter portions 104, 106, 108 of the catheter body. As with the previous example, the lower durometer value catheter distal portion 108 and intermediate portion 106 facilitate the delivery and navigation of the catheter within the vasculature. For instance, the catheter body 102 is navigable through tortuous vasculature. The relatively higher durometer value of the catheter distal portion (and to a lesser extent the intermediate portion) assists in providing pushability and torqueability to the catheter body 102.

For example, where the catheter body 102 includes three or more durometer values, as described above, the catheter body 102 is formed of polyurethane or PEBAX with the catheter proximal portion 104 having a durometer DP of Shore hardness A-A2, the catheter intermediate portion 106 having a durometer DI of B1-B2, and the catheter distal portion 108 having a durometer DD of C1-C2, where DP>DI>DD. Stated another way, the catheter body 102 has a gradually decreasing durometer value (and corresponding stiffness) from the catheter proximal portion 104 to the catheter distal portion 108.

Optionally, the catheter body 102 with the multi-durometer value construction is formed by a co-extrusion process. In one example, a Total Intermittent Extrusion (TIE) process is used. In a TIE process two or more different durometer value polymer resins are extruded from separate dies in line, with the higher durometer value polymer used for the proximal end of the catheter body (e.g., the catheter proximal portion 104) and the lower durometer value polymer used for the distal end of the catheter body (e.g., the catheter distal portion 108), with an intermediate transition zone therebetween, such as the intermediate portion 106 of the catheter body 102. As discussed previously, in one example a 6233 PEBAX is used for the distal end and a 7233 PEBAX is used for the proximal end. In one example, the extruded catheter profile is homogenous along the length of the catheter with the durometer value of the catheter varying along the length. That is to say, the catheter materials are gradually mixed in various amounts according to the desired durometer value and thereafter extruded. In another example, varying of the catheter body 102 durometer value includes extruding one of the proximal and distal portions 104, 108 (e.g., the materials having one of the higher or lower durometer values, respectively) in an end to end fashion and then switching the extrusion resin to a lower or higher durometer material, for the distal and proximal portions 108, 104, respectively.

In other examples, the catheter profile can include two or more layers of material. For example, in one embodiment, the transition zone between the distal end and the proximal end can include a mix of material as the durometer values change from the 6233 PEBAX to the 7233 PEBAX. Stated another way, multiple layers of differing durometer materials are coextruded and alternatively interrupted or added to provide the desired durometer value for the overall catheter body 102.

In still other examples, the catheter body 102 is formed with other processes as known to those of skill in the art, including, but not limited to, shrinking tubing along a lumen liner, welding catheter tubes with varying diameter together at junctions and the like.

FIG. 2 shows a cross-section of the thrombectomy catheter 100, in accordance with one embodiment of the present disclosure. In this example, the infusion lumen 111 is located off-center relative to the aspiration lumen 110 with a septum 202 separating the infusion lumen 111 from the aspiration lumen 110. The catheter body 102 includes an exterior catheter surface and an interior catheter surface, and the aspiration lumen 110 is circumscribed by the interior catheter surface. As shown, exterior catheter surface is featureless and the infusion lumen 111 is recessed relative to the exterior catheter surface. The recessed infusion lumen 111 facilitates the delivery and navigation of the catheter body 102 by providing an isodiametric cylindrical profile, in one example. Further, the recessed infusion lumen 111 includes a partial profile within the aspiration lumen 110 formed by the infusion lumen sidewall. The infusion lumen 111 is positioned at the perimeter of the aspiration lumen 110 to ensure the largest overall profile is available for aspiration of thrombus particles through the aspiration lumen without interference by an infusion lumen, for instance an infusion lumen positioned centrally with the aspiration lumen or resting along an infusion lumen wall (as with a lumen infusion sidewall separate from an aspiration lumen sidewall).

In one embodiment, the catheter body 102 has a diameter of 6 French (Fr) and is inserted using a 0.014 inch guide wire. In another embodiment, the catheter body 102 has a diameter of 8 French and uses a 0.014 inch to a 0.035 inch guide wire for insertion. Optionally, the catheter body 102 includes other diameters and is accordingly usable with corresponding guide wires for delivery.

In one example, the catheter body 102 has a homogenous cross-sectional profile. In other words, the cross-section profile of the catheter body 102, including the infusion lumen 111 and the aspiration lumen, is formed simultaneously and is correspondingly without any sort of bond line or weld line between the sidewall of the infusion lumen 111 and the sidewall of the aspiration lumen 110. This contrasts to a structure where the two lumens are formed separately and then bonded together at a later stage. The homogenous cross-section of the catheter body 102 provides for a more robust structure that is resistant to fracture or peeling of one lumen relative to the other lumen since any bending or torquing of the catheter or the pressures within the lumens will not cause a rupture of a bond line between the two lumens. Alternatively, the aspiration and infusion lumens 110, 111 are separately formed and thereafter coupled together for instance, with welds, adhesives, reflowing and the like.

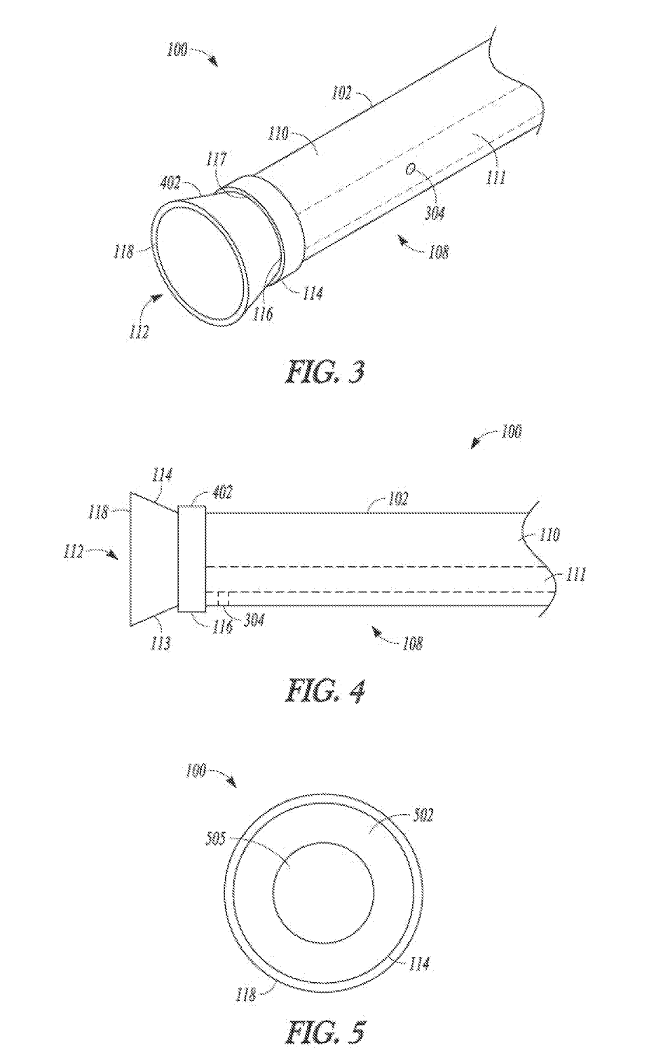

FIG. 3 shows a perspective view of the distal portion 108 of the thrombectomy catheter 100, in accordance with one embodiment. FIG. 4 shows a side view of the distal portion 108 of the thrombectomy catheter 100. As shown in each of these examples, the distal portion includes an aspiration orifice 112 and an infusion orifice 304. As described herein, the aspiration and infusion orifices 112, 304 cooperate during a thrombectomy procedure to hydrodynamically remove thrombus from a vessel, macerate the thrombus and aspirate the thrombus from the vessel.

### Infusion System

Referring again to FIGS. 3 and 4, the infusion lumen 111 extends along the catheter body 102 toward the distal portion 108 with the infusion orifice 304 extending through the catheter body (e.g., through a sidewall of the catheter body adjacent to the infusion lumen 111) to direct a fluid jet away from the catheter body 102. As described herein, the infusion lumen 111 is fluidly coupled with a fluid source configured to provide pressurized fluid, such as saline, for instance at a pressure of around 1500 psi or less. The pressurized fluid is delivered through the infusion orifice 304 and is metered by the orifice 304 to form the fluid jet for the thrombectomy procedure.

In one embodiment of the present disclosure, a single infusion orifice 304 is provided that is configured to direct a fluid jet radially away from a longitudinal axis of the catheter body 102. For instance, the single infusion orifice 304 is directed away from the catheter body 102 to ensure the fluid jet generated at the infusion orifice impinges upon thrombus in a vessel surrounding the catheter body 102. By rotating the catheter body 102 (for instance a catheter body including a higher durometer value proximal portion 104), the infusion orifice 304 and the corresponding fluid jet travel the full measure of the vessel and can thereby remove all thrombus around the catheter distal portion 108. In one example, the infusion orifice 304 has a diameter of about 0.009 inches. In another example the infusion orifice 304 has a diameter of about 0.012 inches. In still another example, the infusion orifice has a diameter in the range of around 0.007 to 0.014 inches. Optionally, the infusion orifice 304 has a diameter configured to generate a fluid jet having a desired velocity and fluid flow rate according to the source of pressurized fluid (e.g., the pressure and flow rate for a pump system coupled with the catheter body 102). Stated another way, the infusion orifice 304 shape and size are configured to cooperate with a fluid source to provide a fluid jet with desired velocity and flow rate values.

In the example described above, a single infusion orifice 304 is provided. In other examples, a plurality of infusion orifices 304 are provided at one or more locations on the catheter body 102 (e.g., radially around the catheter distal portion 104, longitudinally, and the like). A single infusion orifice 304, as shown in FIG. 4 concentrates the hydrodynamic energy of the infusion fluid to better break up the thrombosis. That is to say, by using a single infusion orifice 304, even a low pressure fluid source (for instance, 1500 psi or less having a low flow rate of 1 to 3 cc) is used to generate a fluid jet at the orifice 304 with sufficient hydrodynamic energy to perform a thrombectomy procedure normally reserved for fluid sources providing fluid at high pressure (e.g., 10,000 psi or more). The concentrated fluid jet at the infusion orifice 304 may then be traversed around the body vessel to provide similar efficacy to high pressure thrombectomy treatments using catheters that have a plurality of jet orifices and robust construction sufficient to deliver high pressure fluids.

Different embodiments of the thrombectomy catheter 100 use different infusion fluid flow rates. One example catheter uses a flow rate of about 1.5 cc/sec to provide a fluid jet at the infusion orifice 304 configured to remove and macerate thrombus. Another example uses about 2 cc/sec. Still another example uses about 3 cc/sec. As described above, the velocity of and flow rate of the infusion fluid leaving the infusion orifice 304 is dependent on the flow rate and pressure of the fluid source and the size and shape of the infusion orifice 304. As discussed herein below, a low pressure fluid source, such as a medication or contrast injector is used as the fluid source for the thrombectomy catheter 100. The thrombectomy catheter 100 described herein with the infusion orifice 304 and infusion lumen 111 thereby provides a thrombectomy system configured to effectively remove and macerate thrombus while using low pressure and low flow rate (e.g., medication and contrast) injectors and does not necessarily require high pressure fluid sources otherwise used with other thrombectomy procedures.

The infusion lumen 111 and the infusion orifice 304 are configured, in one example, to mitigate hemolysis, the destruction of blood cells through hydrodynamic energy. The present system constrains the infusion velocity within a range of from about 20 msec to about 30 msec to mitigate hemolysis. The infusion orifice 304, in one example, is sized and shaped to cooperate with the flow rate through the catheter (and accordingly cooperates with the pressurized fluid source) to ensure the infusion velocity at the orifice 304 is between around 20 msec to about 30 msec. By concentrating the infusion flow through the infusion orifice 304 having a specified diameter and shape and a single location on the catheter body 102, the infusion velocity is readily controllable while at the same time providing a localized jet of infusion fluid for maceration of thrombus.

### Aspiration System

In this example, the aspiration lumen 110 includes an aspiration orifice 112 that is open at a distal end 113 of the catheter body 102. A radiopaque collar 402 is in one example located on the distal portion 108. The radiopaque collar assists with imaging of the catheter distal portion 108 during insertion and navigation through a vessel, under fluoroscopic viewing.

In one example, the distal end 113 includes a widened aspiration orifice distal member 114 (e.g., a wide mouth portion providing a larger profile than an adjacent portion of the catheter body 102). The widened aspiration orifice distal member 114 includes a proximal portion 116 coupled to an end 117 of the catheter body 112 as shown in FIG. 3. The widened aspiration orifice distal member 114 includes an opening at the proximal portion 116 sized similar to the aspiration lumen 110. The distal end 118 of the widened aspiration orifice distal member 114 includes an opening wider than the aspiration lumen 110. The widened aspiration orifice distal member 114 is attached to the end of the catheter body 102 by one or more of heat bonding, welding, adhering, reflowing and the like. This widened, funnel-shaped, distal member 114 (e.g., a wide mouth feature) provides for improved aspiration, as will be further discussed below.

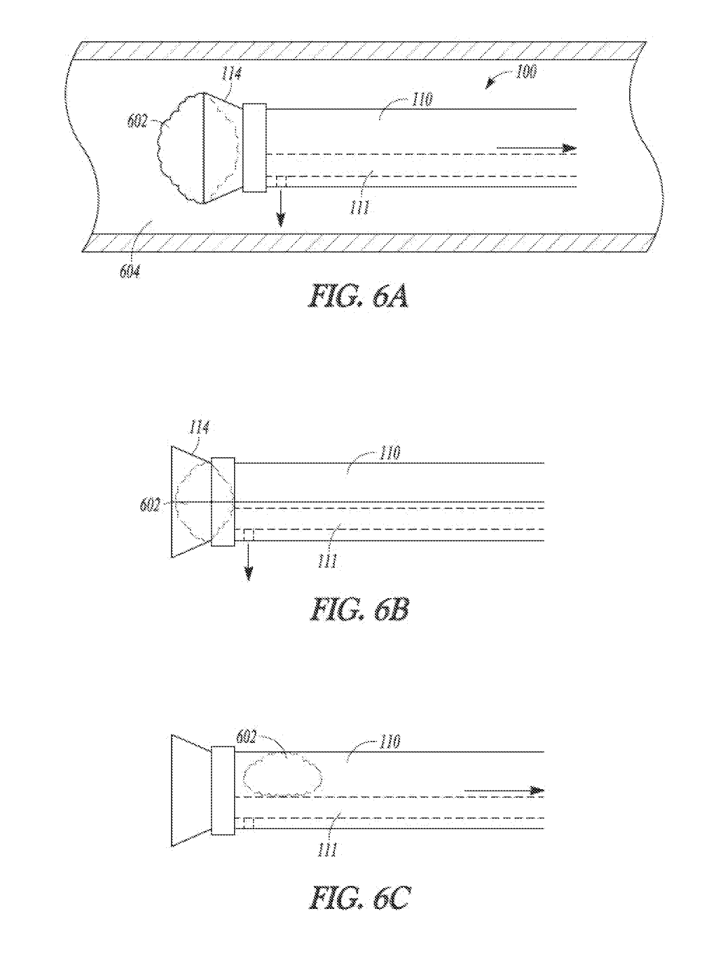

FIG. 5 shows an end view of the widened aspiration orifice distal member 114, unattached to the catheter body. FIG. 6A shows a side view of the thrombectomy catheter 100 in a vessel 604 with the widened aspiration orifice distal member 114 coupled with the catheter 100 and engaged with thrombus 602.

Referring again to FIG. 5, the wide mouth of widened aspiration orifice distal member 114 defines an inner sloping surface 502 that extends from the distal end 118 inward to a proximal portion of the widened aspiration orifice distal member 114 that is attached to the catheter body 104 and communicates with the aspiration lumen 110. In one example, the interface between the widened aspiration orifice distal member 114 and the aspiration lumen 110 is relatively smooth or flush to facilitate the transition of thrombus form the widened aspiration orifice distal member 114 to the aspiration lumen 110.

As shown in FIGS. 5 and 6A, the volume within the widened aspiration orifice distal member 114 from its tip to its connection with the catheter body 102 is free from structural obstructions. Stated another way, the inner sloping surface 502 is substantially continuous and thereby without any interruptions (e.g., humps, projections and the like). That is to say, the distal end 118 of the distal member 114 is substantially continuous (e.g., without obstructions) at the distal end and proximal to the distal end. Accordingly, as thrombus 602 is aspirated into the catheter, the thrombus becomes wedged at the distal end of, or within the distal member 114. The funnel shape of the distal member 114 then seals against the thrombus, and the aspiration pressure of aspiration lumen 110 continues vacuuming and collapsing the thrombus 602 into the gradually narrowing widened aspiration orifice distal member 114 to break it up into smaller pieces that can then fit within and be transferred down the aspiration lumen 110.

FIGS. 6B and 6C show further details of a thrombus being aspirated with FIG. 6B showing a side view of the thrombectomy catheter 110 with the thrombus 602 collapsed within the wide mouth distal member 114 and translated toward the proximal catheter end. FIG. 6C shows a side view of the thrombectomy catheter 110 with the thrombus 602 collapsed (or broken up) into the smaller diameter portion of the catheter 110 and translated toward the proximal catheter end for disposal.

The present wide mouth shape reduces any occurrence of fluid diversion around gaps (e.g., leaks) between the thrombus and the aspiration lumen, which reduce the aspiration pressure (e.g., vacuum) incident on thrombus within the widened aspiration orifice distal member 114. That is to say, the unobstructed annular shape of the widened aspiration orifice distal member 114 allows thrombus to seat along the member and substantially prevents the formation of gaps between the thrombus, and projecting features within the distal member 114. Fluid leaks around the thrombus are thereby substantially minimized and the full vacuum of the aspiration lumen 110 is applied to the thrombus.

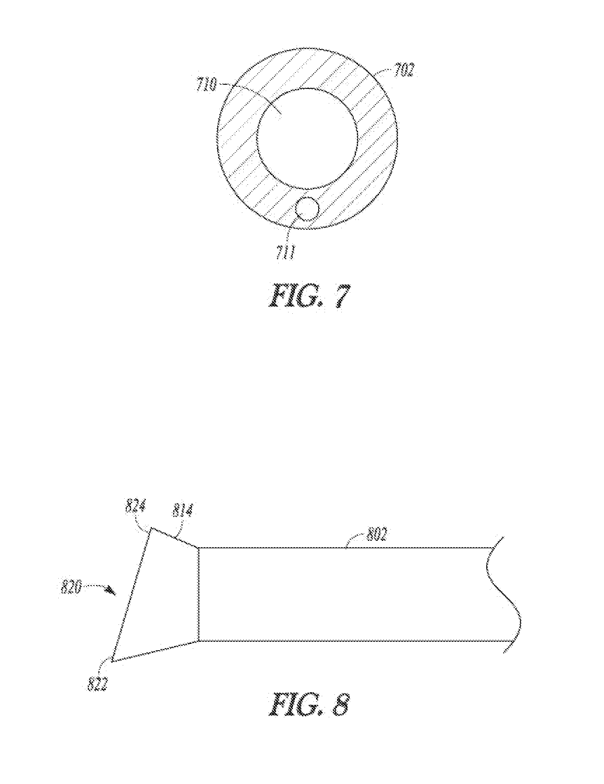

FIG. 7 shows a cross-section of a thrombectomy catheter 702 with the infusion lumen 711 fully positioned with the sidewall of the catheter. The provision of the infusion lumen 711 ensures the profile of the aspiration lumen 710 is substantially isodiametric. In some examples, the isodiametric aspiration lumen 710 (free of obstructions) facilitates the suction and transport of thrombus through the lumen. Alternatively, the catheter 702 includes a portion of the catheter, for instance at the distal or proximal portion 108, 104 that includes the infusion lumen 711 within the sidewall of the catheter while another portion of the catheter, such as the proximal or distal portion 104, 108, includes another part of the infusion lumen 711 partially presented within the aspiration lumen (as shown in FIG. 2) The catheter 702 thereby includes an isodiametric aspiration lumen 710 free of obstructions where needed to efficiently deliver thrombus proximately through the aspiration lumen.

FIG. 8 shows a distal end of a thrombectomy catheter 802, in accordance with another embodiment. In this example, the end of a wide mouth distal member 814 has a beveled shape 815. The beveled shape of the widened aspiration orifice distal member 814 assists with device insertion and navigation into a vessel. The beveled shape 815 tapers from a distal tip 822 slanting up to a proximal portion 824. An aspiration orifice 820 of the widened aspiration orifice distal member 814 accepts thrombus. The beveled shape performs similar to the wide mouth distal member 114 as described above. Accordingly, the widened aspiration orifice distal member 814 is free from any structural obstructions, seats annularly against thrombus within the distal member 814 and forms a seal against any thrombus to prevent fluid diversion around gaps between the thrombus and the aspiration lumen.

### Pressurized Fluid Delivery System

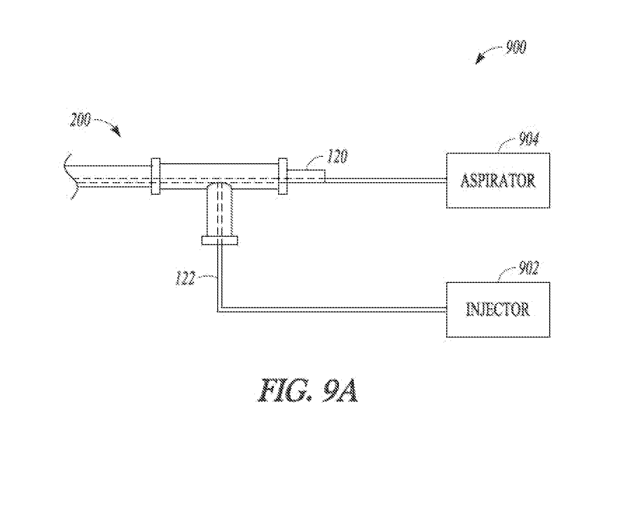

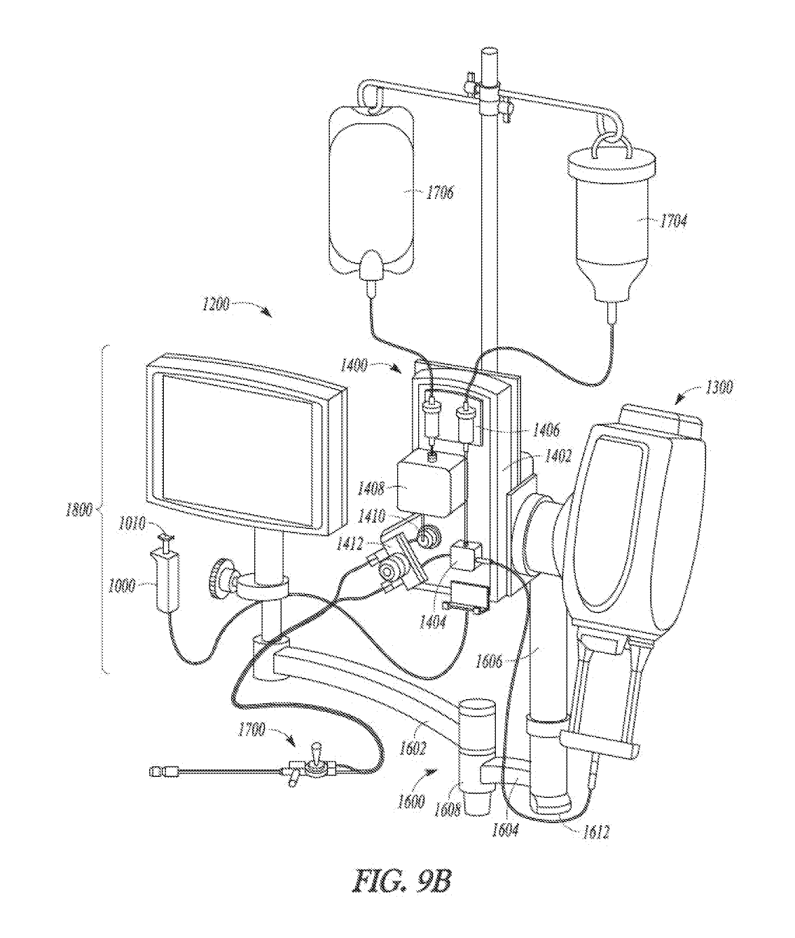

FIG. 9A shows a portion of a thrombectomy system 900, in accordance with one embodiment. The thrombectomy system 900 includes the thrombectomy catheter 100 shown in FIG. 1 with the side port 122 coupled to a fluid delivery device, such as injector 902, and the central port coupled to an aspirator 904, such as a vacuum source.

In use, the thrombectomy catheter 100 is inserted into a vessel using a guide wire, for example. The distal portion 108 of the thrombectomy catheter 100 is navigated through the vasculature placed adjacent a thrombus location. The injector 902 is set to deliver infusion fluid at about 1 cc/s, 1.5 cc/s, 2 cc/s, or 3 cc/s and the like, for example. The injector 902 includes, but is not limited to, a low pressure injector configured for one or more of contrast or medication delivery. A low pressure fluid source is configured to provide infusion fluid to the thrombectomy catheter 100 (802) in a range of between around 300 psi to 2000 psi. As discussed above, the infusion lumen 111 and the infusion orifice 304 are configured by way of shape and diameter to provide a fluid jet having desired flow characteristics (e.g., velocity and flow rate) configured to remove and macerate thrombus according to these lower fluid pressures provided by the injector 902 (as well as lower flow rates compared to high pressure and high flow fluid sources used in other thrombectomy procedures). As discussed herein, the provision of a single infusion orifice 304 localizes the fluid jet to a single location and allows for the use of lower pressure fluids while still removing thrombus. Stated another way, the single infusion orifice 304 avoids the pressure drop across multiple jet orifices, and instead concentrates the hydrodynamic energy provided the low pressure injector 902 at a single location. Other examples can use other fluid delivery devices such as hand-held injectors, high pressure injectors (e.g., 10,000 psi) and the like. The thrombectomy catheter 100 described herein with infusion orifice 304 and infusion lumen 111 provides a thrombectomy system configured to effectively remove and macerate thrombus while using low pressure and low flow rate (e.g., medication and contrast) injectors including continuous delivery pumps without requiring expensive and dedicated high pressure fluid sources (e.g., pumps, injectors and the like).

As the infusion fluid removes and breaks up thrombus, the aspirator 904 coupled with the aspiration lumen 110 is activated to aspirate the particles. The aspirator 904 include a vacuum source, such as a vacuum syringe, vacuum pump and the like.

Another embodiment of an injector system 1200 usable with the present system such as the Medrad Avanta® injector system, is illustrated in FIG. 9B. This example uses a control module 1400, and a powered injector 1300 to which a syringe is connected. The fluid control module 1400 is associated with the injector 1300 for controlling fluid flows delivered by the injector 1300. The fluid control module 1400 is generally adapted to support and control a fluid path set used to connect a syringe associated with the injector 1300 to a catheter (not shown) to be associated with a patient. A source of saline 1706 is in fluid connection with a peristaltic pump 1408.

The fluid delivery system 1200 further includes a support assembly 1600 adapted to support the injector 1300 and the fluid control module 1400, as discussed further herein. The support assembly 1600 may be configured as a movable platform or base so that the fluid delivery system 1200 is generally transportable, or for connection to a standard hospital bed or examination table on which a patient will be located during an injection procedure. Additionally, the fluid delivery system 1200 preferably further includes a user-input control section or device 1800 for interfacing with computer hardware/software (i.e., electronic memory) of the fluid control module 1400 and/or the injector 1300. The fluid control module 1400 generally includes a housing 1402, a valve actuator 1404 for controlling a fluid control valve, a fluid level sensing mechanism 1406, a peristaltic pump 1408, an automatic shut-off or pinch valve 1410, and an air detector assembly 1412.

As indicated, the fluid control module 1400 is generally adapted to support and control the fluid path set 1700 used to connect a syringe associated with the injector 1300 to a catheter (not shown). In a general injection procedure involving the fluid delivery system 1200, the injector 1300 is filled with fluid from the primary fluid container 1704 and delivers the fluid via the fluid path set 1700 to the catheter and, ultimately, the patient. The fluid control module 1400 generally controls or manages the delivery of the injection through a valve associated with the fluid path set 1700, which is controlled or actuated by the valve actuator 1404 on the fluid control module 1400.

The fluid control module 1400 is further adapted to deliver the fluid from the secondary fluid container 1706 under pressure via the peristaltic pump 1408 on the fluid control module 1400. In one embodiment, a handheld controller 1000 includes a plunger or stem control 1010 that, when in a first/low pressure mode, is depressed by the operator to control the flow of fluid from syringe 1300. The farther plunger 1010 is depressed, the greater the flow rate (via, for example, a potentiometer such as a linear potentiometer within the housing of controller 1000). In one embodiment, the operator can use graphical user interface display to change the mode of plunger 1010 to a second mode in which it causes injector 1300 to initiate a high pressure injection as preprogrammed by the operator.

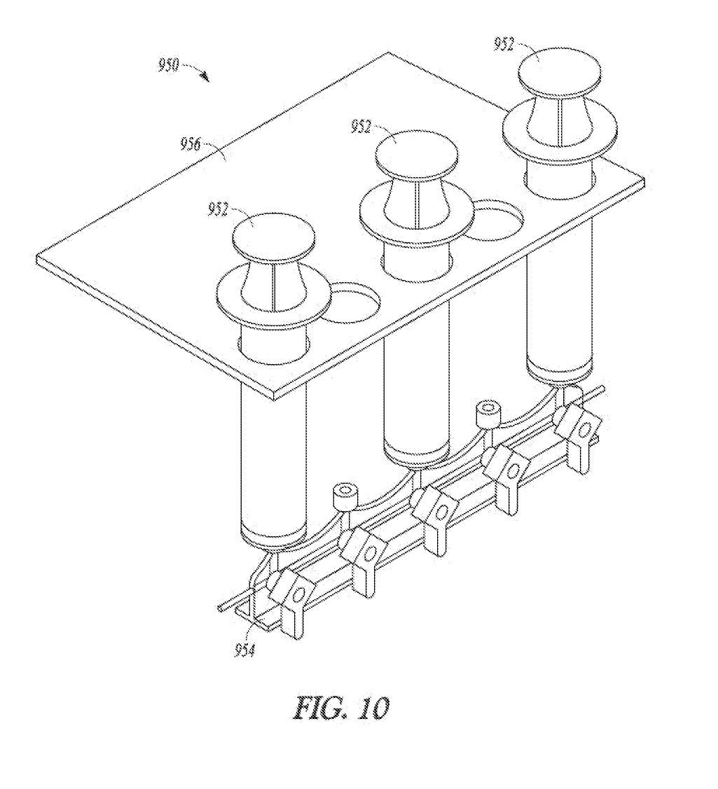

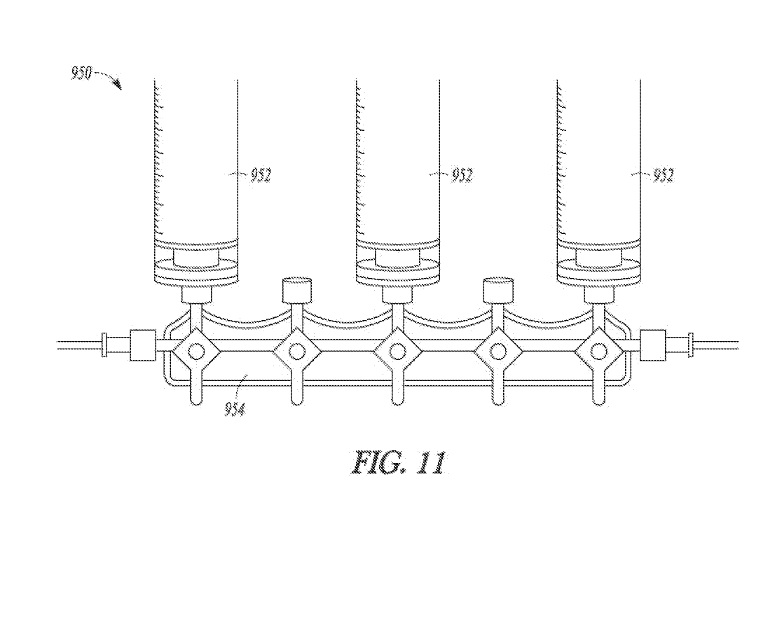

FIG. 10 shows a perspective view of a vacuum source 950, in accordance with one embodiment. FIG. 11 shows a front view of the vacuum source 950.

In this example, the vacuum source 950 is a resettable vacuum source. In one example, the present system described above infuses via a saline filled automated contrast injector with a syringe volume of 150 cc. In the example, an aspiration volume of similar size is used with the aspiration style device (e.g., the vacuum source 950). For example, if a standard 30 cc syringe were used with the injector 902, then the procedure would stop when a corresponding 30 cc syringe of the vacuum source 950 was full to avoid the net subtraction or addition of fluid to the anatomy.

In the example shown in FIGS. 10 and 11 the vacuum source 950 includes a series of 60 cc syringes 952 ganged together via a stop cock style manifold 954. Those of skill in the art would appreciate that varying numbers and sizes of syringes can be used. Optionally, the multiple syringes 952 have more capacity than the infusion source (e.g., the injector 902) and are all resettable prior to any procedure. A frame holder 956 is attached to the vacuum source assembly 950 in one example to keep the syringes 952 upright and visible (and correspondingly hands free). Any number of syringes 952 may be utilized depending on the size of the manifold 954 and the desired aspiration (and injector volume).

In use, the vacuum source 950 (e.g., the aspirator 904) is attached via a luer connector to the thrombectomy catheter 100 and one or more of the stopcocks are opened. After the aspirator 904 is turned on, the aspirated material funneled into the catheter 100, for instance through the widened aspiration orifice distal member 114 and thereafter delivered down the aspiration lumen 110, enters the one or more syringes 952 that have been opened. After one or more of the syringes are filled additional syringes 952 are opened if additional aspiration is needed. If the procedure is complete, the syringes 952 are closed, such as with the stopcock manifold 954, and the syringes 952 are replaced or cleaned as needed for the next procedure.

### Injection Systems

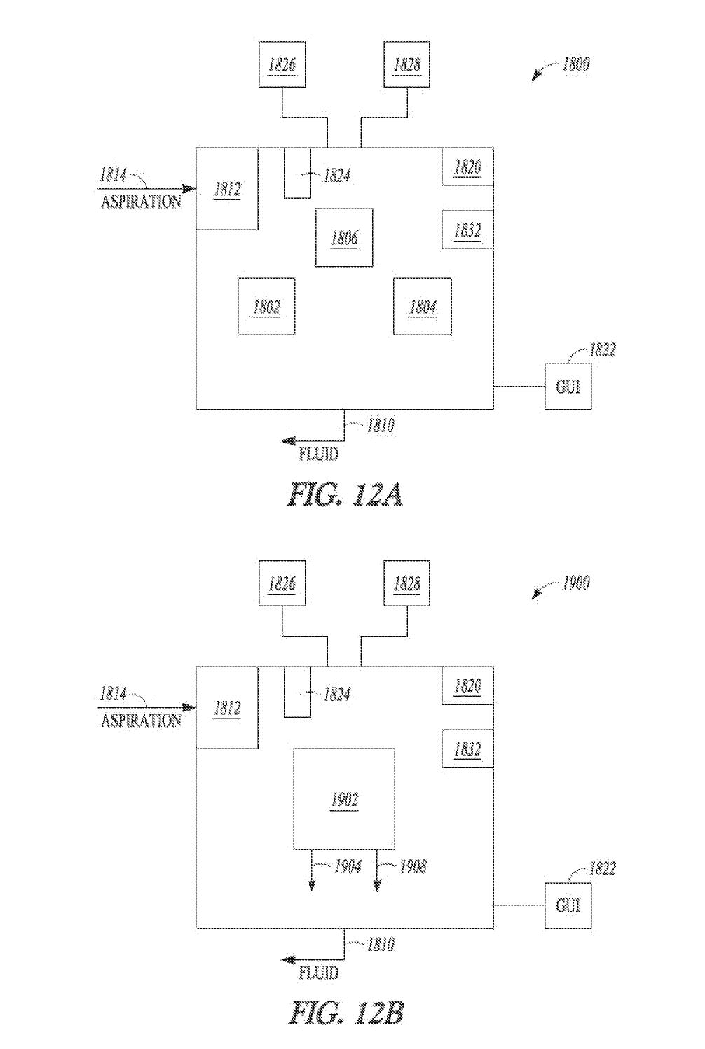

FIG. 12A shows a schematic view of one example of an injector system 1800, according to one embodiment of the present disclosure. FIG. 12B shows a schematic view of another example of an injector system 1900, according to one embodiment of the present disclosure.

These injector systems 1800, 1900 are fluid management mechanisms that can be used with various diagnostic and interventional catheters. The systems incorporate various fluid delivery and management capabilities.

Referring to FIG. 12A, injector system 1800 includes a high-pressure single piston pump 1802. This pump 1802 is configured to provide high-pressure fluid delivery for standard thrombectomy catheters, for example. Some examples provide pressures of about 5,000 psi to about 10,000 psi.

System 1800 further includes a multi-piston pump 1804. Multi-piston pump 1804 is configured to provide medium/low pressure flow for contrast delivery for imaging, flushing agents, and fluid that would be employed using the thrombectomy catheter 100 discussed above. Multi-piston pump 1804 is configured to pump contrast and saline at about 1500 psi and flows of up to 50 ml/sec. Some options have a delivery pressure of about 1000 psi. Some can range from 500 psi to 2500 psi. Pump 1804 is a continuous flow pump (i.e. it does not have to refill like a syringe pump).

One option further includes a single piston pump 1806. Pump 1806 is a pump configured to pump contrast or saline at 1500 psi and flows of up to 50 ml/sec, but it must be refilled. In some embodiments of system 1800, pump 1806 is omitted or pump 1804 is omitted.

Each of pumps 1802, 1804, and 1806 are operatively coupled to an outlet fluid line 1810 to deliver fluid to a catheter or other tool. Pumps 1802, 1804, and 1806 are designed to operate independently, in that only one pump would deliver-fluid at one time.

System 1800 further includes an aspiration module 1812. Aspiration module 1812 is configured to withdraw fluids through either the fluid delivery catheter or a separate catheter.

Each of pumps 1802, 1804, and 1806 are configured to share a common architecture. For example, system 1800 can optionally include operating an power system 1820, a graphical user interface (GUI) 1822, a fluid assurance/air detection module 1824, and one or more bulk fluid sources 1826, 1828. On option includes a module 1832 configured to provide fluid mixing dynamically and monitoring remaining volumes of fluid 1826, 1828. Some options further provide for multi-use disposable, interface and informatics connectivity, and catheter/disposable recognition.

In different embodiments, certain features discussed above are combined in different ways. One example configuration combines pumps 1802 and 1804 with aspiration module 1812, and at least one or more of a standard thrombectomy catheter, a thrombectomy catheter 100 or a diagnostic catheter. Another example configuration combines pumps 1802 and 1806 with the aspiration module 1812, and at least one or more of a standard thrombectomy catheter, thrombectomy catheter 100 or a diagnostic catheter. Still another example configuration combines the pump 1804 with the aspiration module 1812 and one or more of thrombectomy catheter 100 or a diagnostic catheter. An additional configuration combines pump 1806 with aspiration module 1812 and one or more of thrombectomy catheter 100 or a diagnostic catheter. Yet another example configuration combines pump 1806 and one or more of thrombectomy catheter 100 or a diagnostic catheter. Another configuration includes a single pump piston 1806 and is capable of working with a contrast injector or with the thrombectomy catheter 100 discussed above. Conversely, the first described configuration has more complexity because it is compatible with all catheters and capabilities.

By providing all the different capabilities in one compact system, fluid injection system 1800 can be used for multiple cases. Typical injection systems are either high-pressure or low-pressure and so a medical staff must have both systems and be capable of using both. By combing the systems, injector system 1800 is more likely to be used as the set-up is minimal and the learning curve is reduced. Additional benefits include time savings, reduced consumables, additional floor space and availability of a device for any procedure.

Referring to FIG. 12B, injector system 1900 can include any of the features discussed above for injector system 1800, and those features will not be discussed. Similarly, the same multiple use configurations utilizing various catheters can also be utilized.

Here, injector system 1900 includes a multi-piston pump 1902 that is capable of delivering low pressure fluids 1904 for use in contrast imaging, flushing solutions, or use with thrombectomy catheter 100 discussed above. Further multi-piston pump 1904 can deliver high pressure fluids 1908 for use with standard thrombectomy catheters.

Again, by providing all the different capabilities in one compact system, fluid injection system 1900 can be used for multiple cases. Typical injection systems are either high-pressure or low-pressure and so a medical staff must have both systems and be capable of using both. By combing the systems, injector system 1900 is more likely to be used as the set-up is minimal and the learning curve is reduced. Additional benefits include time savings, reduced consumables, additional floor space and availability of a device for any procedure.

### Thrombectomy Catheter

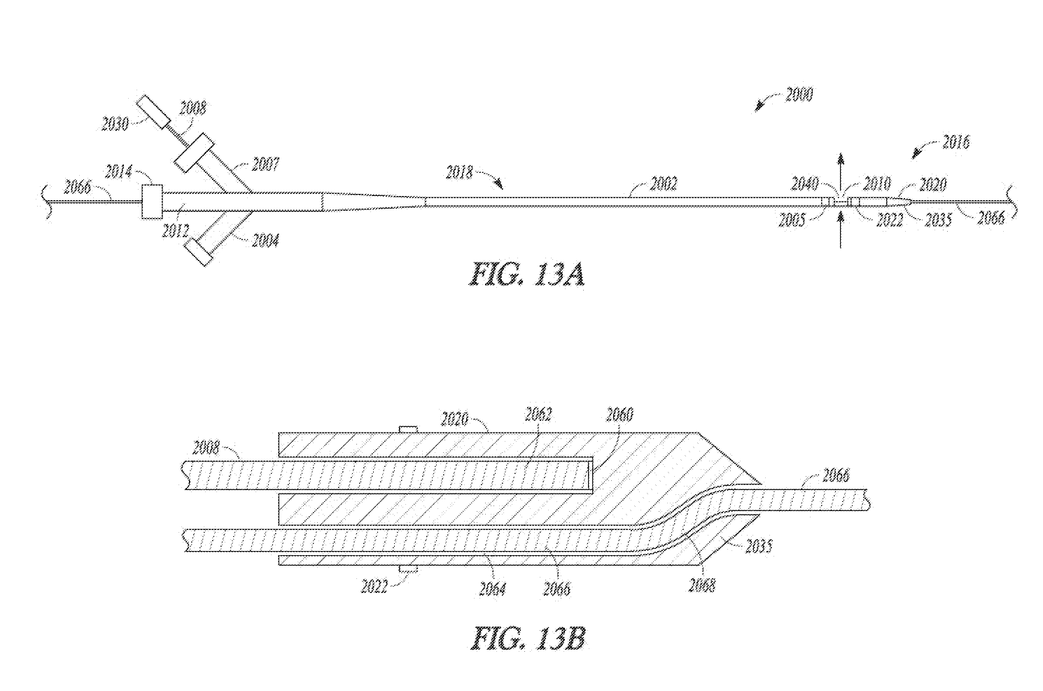

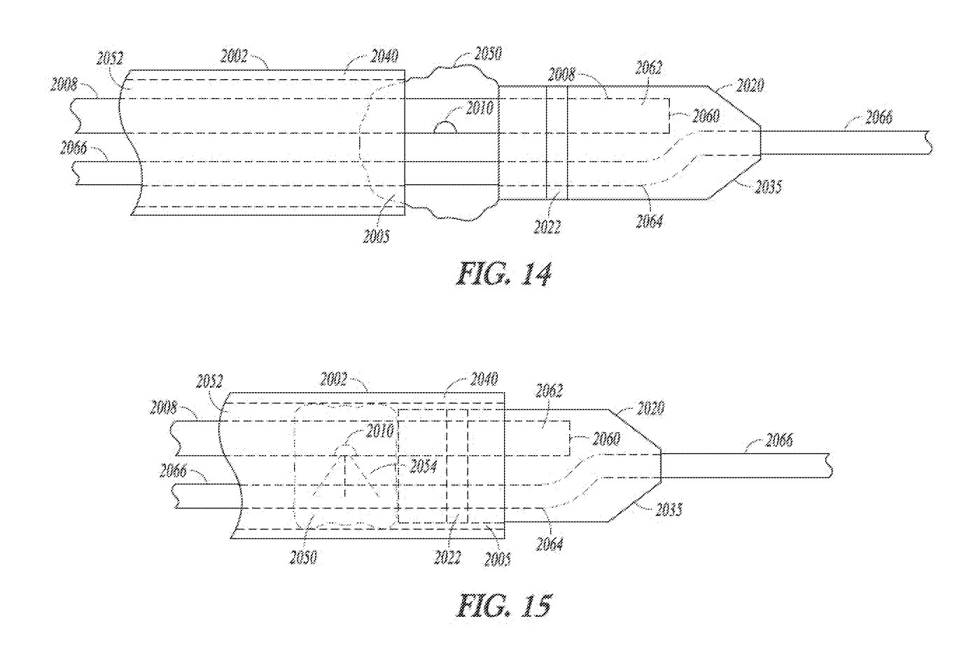

FIG. 13A shows a side view of an embodiment of a thrombectomy catheter 2000 according to one or more embodiments of the present disclosure. Thrombectomy catheter 2000 generally includes a catheter body 2002 which includes an aspiration lumen 2052 (see FIGS. 14 and 15) extending though the catheter body 2002 and open at a distal end at an aspiration orifice 2005. The aspiration lumen 2052 communicates with an aspiration port 2004 which can be coupled to a vacuum source as discussed above, for instance with a hemostasis valve, fitting or the like. The vacuum source includes, but is not limited to, a syringe, vacuum bottle, roller pump, vacuum pump or the like. The thrombectomy catheter 2000 includes a fluid injection port 2007 (similarly including a hemostatis valve, fitting or the like). In this example, fluid can be delivered through an infusion body 2008, such as a stainless steel hypotube, polymer tube, Nitinol tube or the like. Infusion body 2008 can include a connection member 2030 for connection to an injector source, such as the injectors discussed above. Infusion body includes an internal lumen extending through the infusion body and having an infusion orifice 2010 near a catheter distal portion 2016. In one example, a single infusion orifice 2010 is used. Infusion body extends through the catheter body 2002 within the aspiration lumen 2052.

FIG. 13A further shows a guide wire 2066 extending through the catheter body 2002 and an expanded member (2020, described below). The guide wire 2066 facilitates navigation through the vasculature and further allows for sliding movement of the components of the thrombectomy catheter relative to one another while maintaining coincidence of the infusion body 2008 (and the expanded member) relative to the catheter body 2002. As shown in FIG. 13A, the guide wire 2066 extends through a manifold 2012 coupled with a catheter proximal portion 2018. The guide wire 2066 enters the manifold 2012 through an access port 2014. As with the other ports, including the aspiration port 2004 and the fluid injection port 2007, a hemostasis valve is optionally provided at the access port 2014 to facilitate the sealed delivery of the guide wire 2066 through the manifold 2012. The sealed environment provided within the thrombectomy catheter 2000 allows for aspiration of infusion fluids including entrained particulate from the distal end of the infusion body 2008 and the catheter distal portion 2016 (e.g., adjacent to the expanded member 2020 at the aspiration orifice 2005).

Coupled to a distal end of the infusion body 2008 and located distally from the infusion orifice 2010 is an expanded member 2020. The expanded member 2020 is shown in FIG. 13B as a detailed cross section. The expanded member 2020 includes a diameter that is larger than the infusion body 2008. In one example expanded member 2020 is dimensioned to fit within the aspiration orifice 2005. In one example, expanded member 2020 includes a tapered distal portion 2035 and one or more marker bands 2022. In some examples, the expanded member 2020 includes a glue bulb or an additional coil of wire. In other examples, the aspiration lumen 2052 includes a widened aspiration orifice distal member 114, for instance as shown in FIGS. 3 and 4. Optionally, the expanded member 2020 is tapered near a proximal end to facilitate delivery into the aspiration orifice 2005.

As further shown in FIG. 13B, the expanded member 2020 includes an infusion body recess 2060 sized and shaped to receive an infusion body distal end 2062 (e.g., the distal end of a hypotube providing the infusion fluid to the fluid infusion orifice 2010). In one example the infusion body distal end 2062 is fixedly coupled with the expanded member with at least one mechanism including, but not limited to, adhesives within the infusion body recess 2060, crimping, overmolding, mechanical interference fitting and the like. In another example, the expanded member 2020 is sized and shaped for sliding reception of an instrument, such as a guide wire within a guide wire passage 2064. In FIG. 13B, the guide wire 2066 is shown extending through the expanded member 2020. Optionally, the guide wire passage 2064 includes a passage that is at least partially non-linear as shown, including for instance an elbow 2068. In another option, the guide wire passage 2064 is substantially centrally located within the expanded member 2020. The expanded member 2020 rides over the guide wire 2066 with the guide wire 2066 acting as a rail. In yet another option, the guide wire passage is provided within the infusion body 2008 and accordingly consolidates the guide wire and the infusion body 2008 in a coincident configuration.

The expanded member 2020 is configured to free plugs of material 2050 that are lodged within the aspiration orifice 2005. For example, plugs 2050 of thrombus plug the tip 2040 of the thrombectomy catheter 2000, as shown in FIG. 14. Retraction of the infusion body 2008 (or conversely translational advancement of the catheter tip 2040 past the expanded member 2020) as shown in FIG. 15 frees the blockage, thus restoring aspiration without the need to remove the catheter from the body. Optionally, the expanded member 2020 and the infusion body 2008 are removable from the catheter body 2002, for instance by proximal or distal sliding of the infusion body 2008 relative to the catheter body. In yet another example, the expanded member 2020 and the infusion body 2008 are provided as a unitary device sized shaped for use with one or more standard delivery or interventional catheters having interior lumens sized to receive the infusion body 2008 and the expanded member 2020 therein.

In operation, thrombus plugs the aspiration orifice 2005. The user manipulates the infusion body 2008 by one or more of rotating the infusion body 2008 in either direction (clockwise or counterclockwise) and by reciprocating the infusion body longitudinally relative to the catheter body 2002. As shown in FIGS. 14 and 15 the expandable member 2020 translates as a slidable element relative to the guide wire 2066 received within the guide wire passage 2064. The guide wire 2066 according serves as a rail for the expanded member 2020. The guide wire 2066, also received in the aspiration lumen 2052, assists in centering the expandable member 2020 relative to the aspiration lumen 2052.

When the expanded member 2020 is within the aspiration lumen 2052 it physically pushes (e.g., plunges, mechanically engages and the like) the thrombus 2050 into and down the aspiration lumen 2052. At the same time, the infusion orifice 2010 is positioned inside the aspiration lumen 2052 of the catheter body 2002 and the infusion jet 2054 assists in breaking up the thrombus 2050. The expanded member 2020 acts as a plug for the aspiration orifice 2005 and the infusion jet will be directed toward the blocking thrombus, and the outflow of the infusion jet 2054 from the infusion orifice 2010 will carry the thrombus through the aspiration lumen 2052. Stated another way, the infusion orifice 2010 and the generated infusion jet 2054 cooperate with the mechanical engagement (e.g., plunging) provided by the expanded member to dislodge plugs 2050 of material at the aspiration orifice 2005 and within the aspiration lumen 2052. This combined functionality minimizes and substantially eliminates plugging of the aspiration lumen 2052 even with the delivery of low pressure infusion fluids through the infusion orifice 2010.

In the present example, the expanded member 2020 cooperates with the catheter body 2002 to remove thrombus 2050 in such a manner that the present example can eliminate the wider aspiration orifice distal member 114, discussed above. This allows the device to smoothly track through blockages and vasculature without embolization or vessel damage. In some embodiments, the wider aspiration orifice distal member 114 can be used with the expanded member 2020.

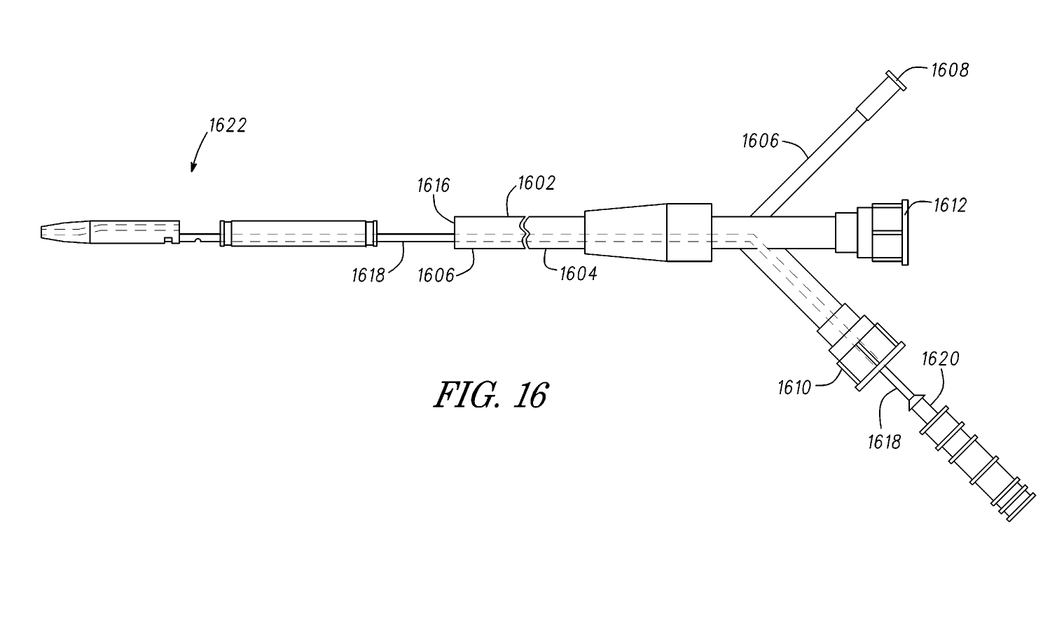

FIG. 16 shows another example of a thrombectomy catheter 1600. As with the previous examples the thrombectomy catheter 1600 includes a catheter body 1602 extending between a catheter proximal portion 1604 and a catheter distal portion 1606. As shown in FIG. 16 the catheter body 1602 is coupled with a manifold 1606. The manifold 1606 includes one or more ports, such as an aspiration port 1608, a guide wire port 1612 and an infusion port 1610. In one example, the thrombectomy catheter 1600 is used to remove thrombus from a vessel within the vasculature of a patient.

The thrombectomy catheter 1600 includes a catheter distal portion 1606 and an exposed infusion body 1618. As will be described herein, in one example the infusion body 1618 includes one or more infusion orifices sized and shaped to provide a flow of infusion fluid from the catheter body 1602. The infusion fluid hydrodynamically engages with thrombus within the vasculature, dislodges the thrombus and in some examples macerates the thrombus into particulate matter. An aspiration lumen 1614 extending through the catheter body 1602 provides a vacuum at an aspiration orifice 1616 that draws the particulate matter (entrained within the infusion fluid) through the aspiration lumen 1614 to the aspiration port 1608. The aspiration port 1608 is coupled with an aspiration pump (e.g., a pump, roller pump, vacuum syringes or the like) configured to draw the entrained particulate matter into a reservoir, effluent bag or syringes for eventual removal. The aspiration pump is in one example a roller pump coupled with an effluent line (coupled with the aspiration port 1608) and optionally housed in drive unit including an infusion pump.

In another example, and as previously described, the manifold 1606 includes an infusion port 1610. As shown the infusion port 1610 includes an infusion body 1618 slideably received therein. The infusion body 1618 extends from the infusion port 1610 through the catheter body 1602 and through the aspiration orifice 1616. In one example the infusion body 1618 is a part of a plug removable assembly 1622 at a distal end of the infusion body 1618. The plug removable assembly 1622 will be described herein. In one example the infusion body 1618 is coupled with an infusion fitting 1620 at the manifold 1606. The infusion fitting 1620 provides a fitting for coupling with one or more pumps for instance a continuous or near continuous flow infusion pump (e.g., pump 1902 or 2400 described herein) configured to provide a flow of infusion fluid through the infusion body 1618 to at least one infusion orifice, for instance an infusion orifice positioned near the catheter distal portion 1606 as described herein.

In another example, and as previously described herein, the manifold 1606 includes a guide wire port 1612. The guide wire port 1612 is in one example substantially coincident with the catheter body 1602. In one example a guide wire is fed through the guide wire port 1612 (e.g., through a hemostasis valve) through the catheter body 1602 and through a corresponding guide wire lumen within an expanded member for instance an expanded member coupled with the infusion body 1618 (further described herein). The guide wire allows for navigation of the thrombectomy catheter 1600 through vasculature including tortuous vasculature. For instance the guide wire is traversed through the vasculature and the catheter body 1600 is thereafter fed over the guide wire to facilitate the tracking of the thrombectomy catheter 1600 to a desired location within the vasculature.

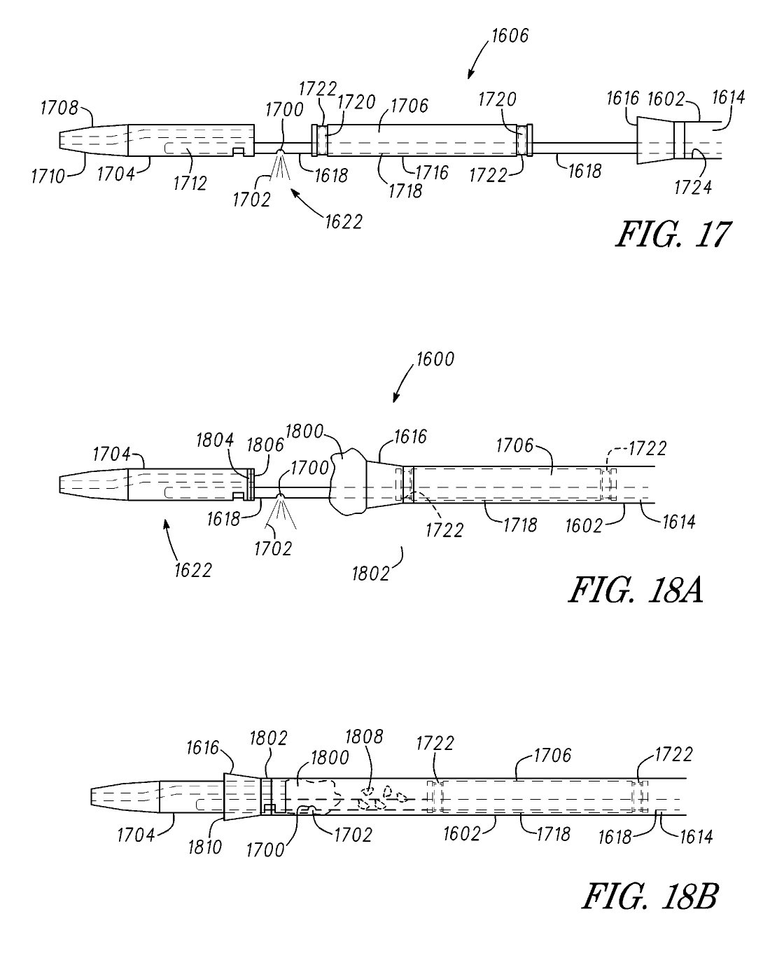

Referring now to FIG. 17 a detailed schematic view of the catheter distal portion 1606 is provided. As shown in FIG. 17 the plug removal assembly 1622 is positioned distally relative to the aspiration orifice 1616. The aspiration orifice 1616 has a wide mouth configuration. For instance the aspiration orifice has a mouth optionally tapering outwardly from the otherwise isodiametric diameter of the catheter body 1602. As previously described herein a wide mouth aspiration orifice, such as the aspiration orifice 1616, provides in one example an enlarged shape sized and shaped to receive one or more plugs of thrombus therein. In one example, a marker band is provided around or near the aspiration orifice 1616. The marker band provided at the aspiration orifice 1616 cooperates with marker bands, for instance marker bands on the guide member 1706 (described herein), to facilitate the accurate positioning of the guide member 1706 and the expanded member 1704 relative to the opening of the aspiration lumen 1614 (the aspiration orifice 1616). Stated another way, the viewing of the relative position of the marker bands ensures that the guide member 1706 is reliably maintained within the aspiration lumen 1614 to ensure tracking of the expanded member 1704 as described herein.

As further shown in FIG. 17, the infusion body 1618 partially extends from the aspiration orifice 1616. Stated another way a portion of the infusion body 1618 is received within the aspiration lumen 1614 while another portion of the infusion body 1618 is presented beyond or distal to the aspiration orifice 1616 (in a deployed position). In this configuration one or more infusion orifices 1700, in an example a single infusion orifice 1700, is shown outside of the aspiration lumen 1614 and directed in a radial manner away from the catheter body 1602 to generate an infusion jet 1702. In one example the infusion jet 1702 is directed radially or laterally relative to the infusion body 1618 (or the catheter body 1602) to provide a radial flow of infusion fluid that hydrodynamically engages with surrounding thrombus to remove the thrombus from the vessel wall and macerate or abrade the thrombus into particulate for eventual aspiration through the aspiration lumen 1614. The one or more infusion orifices 1700 are configured (for instance with an infusion fluid pressure of around 500 to 1500 psi) to generate the infusion jets 1702 with corresponding velocities of around 1 to 300 meters per second.

In operation the infusion body 1618, for instance those portions of the infusion body 1618 including the expanded member 1704 are delivered outside of the aspiration orifice 1616 while the guide member 1706 remains slidably engaged or coupled within the aspiration lumen 1614. For instance the guide member 1706 is slideably coupled along a catheter body inner wall 1724.

Referring now to the plug removal assembly 1622 the assembly 1622 includes, in one example, two or more components. The plug removal assembly 1622 includes an expanded member 1704 and a guide member 1706. As shown in FIG. 17 the expanded member 1704 is positioned distally relative to the infusion orifice 1700. The expanded member 1704 includes an injection molded or machined component formed of a polymer or resin affixed to the infusion body 1618. For instance the infusion body 1618 is received within a reception recess 1712 of the expanded member 1704. In yet another example a guide wire lumen 1708 extends through the expanded member 1704 for instance through an atraumatic tip 1710. As shown in FIG. 17 the guide wire lumen 1708 has a discontinuous linear configuration to provide space for the reception recess 1712 of the infusion body 1618. In another example the guide wire lumen 1708 has a substantially straight linear configuration extending substantially along the middle of the expanded member 1704, and the reception recess 1712 for the infusion body 1618 is provided off center relative to the guide wire lumen 1708. In another example, the infusion body 1618 is coupled along an exterior surface of the expanded member 1704 to accordingly make room for the guide wire lumen 1708 extending substantially through the middle of the expanded member 1704.

As further shown in FIG. 17, the atraumatic tip 1710 has a tapered configuration to facilitate the delivery of the expanded member 1704 through the aspiration lumen 1614 (e.g., to extend past snags, occlusions or the like within the aspiration lumen 1614). Additionally, the atraumatic tip 1710 has the tapered configuration as well as a relatively soft pliable material (e.g., silicone, rubber or the like) for construction to facilitate the delivery of the expanded member 1704 distally from the catheter body 1602, for instance for the deployment of the infusion orifice 1700 and the corresponding infusion jet 1702 therefrom. The atraumatic tip 1710 is configured to provide a pliable feature that when engaged with the vasculature ensures the vasculature is not damaged.

Referring again to FIG. 17 the plug removal assembly 1622 includes the guide member 1706. In one example the guide member 1706 is an elongate feature coupled along the infusion body 1618. In one example and as shown in FIG. 17, the guide member 1706 has a perimeter substantially corresponding to the perimeter of the catheter body inner wall 1724. Accordingly, while the guide member 1706 is positioned within the aspiration lumen 1614 the guide member 1706 is slideable along the aspiration lumen 1614 (e.g., the catheter body inner wall 1724) and thereby automatically guides the expanded member 1704 into a substantially central position relative to the aspiration lumen 1614 even while the expanded member 1704 is outside of the aspiration lumen 1614 (including for instance the aspiration orifice 1616).

In one example, the guide member 1706 is constructed with but not limited to a guide sleeve 1716. For instance the guide sleeve 1716 is a polymer or resin sheath provided along the infusion body 1618. The guide sleeve 1716 includes a sleeve wall 1718 coupled along the infusion body 1618. In one example one or more sleeve rings 1720 are coupled with the infusion body 1618 for instance by welds. The sleeve rings 1720 present a ring type feature extending around a portion of the infusion body 1618 and accordingly extending away from the infusion body. The guide sleeve 1716 is positioned over top of the sleeve rings 1720 to provide a structural support for the guide sleeve 1716 as well as accurate positioning of the guide sleeve 1716 in the desired configuration along the infusion body 1618. One or more marker bands 1722 (configured for observation during fluoroscopy or the like) are then positioned over top of the guide sleeve 1716 as well as the corresponding sleeve rings 1720 to hold the guide sleeve 1716 in a sandwiched configuration between the marker bands 1722 and the sleeve ring 1720.

In another example, the guide member 1706 includes but is not limited to one or more elongate features extending along the infusion body 1618. For instance, the guide member includes, but is not limited to, a solid resin tube coupled along the infusion body 1618 (e.g., with a passage for a guide wire or aspirated fluid), one or more rings coupled in sequence along the infusion body 1618 (to accordingly form a virtual sleeve) or the like.

Referring again to FIG. 17, the guide member 1706 as previously described herein has a corresponding perimeter or configuration (shape) relative to the catheter body inner wall 1724 of the catheter body 1602. The guide member 1706 when received within the aspiration lumen 1614 of the catheter body 1602 thereby provides a guiding or centering function for the plug removal assembly 1622. For instance, the expanded member 1704 even when deployed outside of the aspiration orifice 1616 is centered relative to the catheter body 1602. During translation of the plug removal assembly 1622 (e.g., to externally provide an infusion jet with the infusion orifice 1700 or for removal of a plug in the aspiration orifice 1616) translation of the expanded member 1704 is conducted in a guided smooth fashion as the expanded member 1704, centered relative to the aspiration orifice 1616, is translated into and out of the aspiration orifice 1616 according to the proximal sliding relationship of the guide member 1706 within the aspiration lumen 1614. Stated another way, while the guide member 1706 is slidably received within the catheter body 1602 (e.g., the aspiration lumen 1614) the guide member 1706 provides a proximal guide that centers and ensures receipt of the expanded member 1704 within the aspiration lumen 1614 when moved from the deployed configuration (shown in FIG. 18A). Further, even while the expanded member 1704 is deployed the guide member 1706 ensures that the expanded member 1704 and the infusion orifice 1700 are centrally positioned relative to the catheter body 1602. The guide member 1706 thereby ensures predictable positioning of the infusion orifice 1700 as well as the expanded member 1704 within the vasculature even while these features are deployed outside of the catheter body 1602.

Referring now to FIGS. 18A and 18B the thrombectomy catheter 1600 is shown in two different configurations, a deployed position as shown in FIG. 18A and a retracted position or plunging position shown in FIG. 18B. Referring first to FIG. 18A, the expanded member 1704 is shown in a deployed position relative to the aspiration lumen 1614 and the aspiration orifice 1616. As further shown the infusion orifice 1700 (and the corresponding infusion jet 1702) is also positioned outside of the aspiration orifice 1616 and the aspiration lumen 1614. The infusion orifice 1700 and the corresponding infusion jet 1702 are thereby directed in a radial fashion away from the thrombectomy catheter 1600 into engagement with thrombus adhered to or positioned along a vessel wall. The infusion fluid delivered through the infusion body and the infusion orifice 1700 generates the infusion jet 1702. In one example the infusion jet 1702 is directed radially as shown. In another example, the infusion jet 1702 is generated at another angle for instance distally away from the tip portion of the infusion body 1614 (e.g., through the expanded member 1704).

As further shown in FIG. 18A the guide member 1706 is positioned within the catheter body 1602. For instance, the guide member 1706 is positioned within the aspiration lumen 1614. The expanded member 1704 positioned at the end of the infusion body 1618 is correspondingly centered relative to the catheter body 1602. Stated another way, the guide member 1706 centers the expanded member 1704 in a substantially linear or coincident fashion relative to the catheter body 1602. As shown in FIG. 18A, corresponding translation of either of the catheter body 1602 or the infusion body 1618 relative to the other component correspondingly moves the expanded member 1704 relative to the catheter body 1602. The expanded member 1704 remains aligned (e.g., coincident) with the catheter body 1602 during translation because of the guide function of the guide member 1706 within the aspiration lumen 1614. That is to say, the guide member 1706 perimeter corresponds to the perimeter at the catheter body inner wall 1724 to ensure the expanded member 1704 is aligned with the catheter body 1602 during translation of the expanded member 1704 into and out of the catheter body 1602.

As further shown in FIG. 18A the thrombectomy catheter 1600 is in an operating configuration where the infusion jet 1702 is directed radially away from the thrombectomy catheter 1600. As shown a thrombus particle such as a thrombus plug 1800 has been freed from the vessel wall and is lodged across or at least partially across the aspiration orifice 1616. In this example the vacuum or suction provided through the aspiration lumen 1614 is insufficient to draw the thrombus plug 1800 fully into the aspiration orifice 1616 and the aspiration lumen 1614. Accordingly, the thrombus plug 1800 negatively affects the aspiration of other particulate surrounding the catheter distal portion 1606 and thereby frustrates continued operation of the thrombectomy catheter 1600. The plug removal assembly 1622, including the guide member 1706 as well as the expanded member 1704, is used to remove the thrombus plug 1800 and thereby restore full function to the thrombectomy catheter 1600.

FIG. 18B shows the plug removal assembly 1622 in operation to remove the thrombus plug 1800. As shown in FIG. 18B, the infusion body 1618 is translated relative to the catheter body 1602 to draw the expanded member 1704 as well as the infusion orifice 1700 into the aspiration lumen 1614. As the expanded member 1704 is drawn distally relative to the aspiration orifice 1616 a plunging surface 1806, shown in FIG. 18A, is engaged against the thrombus plug 1800. The plunging surface 1806 of the expanded member 1704 drives or plunges the thrombus plug 1800 into the aspiration lumen 1614 through the aspiration orifice 1616.

As previously described herein the guide member 1706 aligns the expanded member 1704 with the aspiration lumen 1614. Accordingly, upon withdrawal or retraction of the expanded member 1704 the aligned expanded member is readily drawn into the aspiration lumen 1614 through the aspiration orifice 1616. Repeated reorienting of the infusion body 1618 and the expanded member 1704 into position for reception within the aspiration orifice 1616 is accordingly avoided. The plunging surface 1806 of the expanded member 1704 thereby drives the lodged thrombus plug 1800 into the aspiration lumen immediately with retraction of the infusion body 1618 (or proximal movement of the catheter body 1602 relative to the infusion body).

In one example with the expanded member 1704 positioned within the aspiration lumen 1614 the expanded member 1704 closes the aspiration lumen (at the aspiration orifice 1616) with the infusion orifice 1700 within the catheter body 1602. With the infusion orifice 1700 within the catheter body 1602 a flow of infusion fluid is provided through the infusion body 1618 to the orifice. The corresponding infusion jet 1702 is generated internally within the catheter body 1602. The hydrodynamic engagement of the infusion jet 1702 with the thrombus plug 1800 macerates the thrombus plug into particulate 1808 within the catheter body 1602. The particulate is held within the catheter body 1602 and is accordingly not free to move outside of the catheter body 1602 and into the general vasculature of the patient. Instead, the infusion flow from the infusion orifice 1700 cooperates with the aspiration source, for instance an aspiration pump, coupled with the thrombectomy catheter 1600 to draw the particulate 1808 of the thrombus plug 1800 proximally through the aspiration lumen 1614 to an effluent bag coupled with a thrombectomy catheter system. The thrombectomy catheter system including the thrombectomy catheter 1600, the pumping mechanisms for infusion and aspiration and the like.

By closing the aspiration lumen 1614 (e.g., the catheter body 1602) a closed environment is provided for the thrombus plug 1800. Accordingly, the infusion jet 1702 generated within the catheter body 1602 provides a dedicated source of macerating fluid for the thrombus plug 1800 to ensure complete or near complete maceration of the thrombus plug 1800 and ensure delivery of the thrombus proximally through the aspiration lumen 1614 to an effluent bag. After maceration of the thrombus plug 1800, in one example, the thrombectomy catheter 1600 is ready to continue a thrombectomy procedure, for instance by translating the infusion body 1618 relative to the catheter body 1602 to reposition the plug removal assembly 1622, including the expanded member 1704 and the infusion orifice 1700, in an exterior position such as that shown in FIG. 18A corresponding to the deployed position. With the aspiration orifice 1616 now cleared operation of the infusion orifice 1700 for instance to generate the radially directed infusion jet 1702 is resumed to continue the thrombectomy procedure within the vessel. After completion of the thrombectomy procedure the expanded member 1704 as well as the infusion orifice 1700 are withdrawn into the catheter body 1602 again through the guide function provided by the guide member 1706 to facilitate the withdrawal of the thrombectomy catheter 1600 from the vasculature.

As further shown in FIGS. 18A and 18B and previously described in FIG. 17 one or more marker bands 1722 are optionally provided for the guide member 1706. As further shown in FIG. 18A a catheter marker band 1802 is optionally provided on the catheter body 1602. During a procedure, for instance during movement of the expanded member 1704 and the infusion orifice 1700 between the deployed position and the retracted position, a technician uses the relative movement and location of the one or more marker bands 1722 relative to the catheter marker band 1802 to note and index the position of the expanded member 1704 and the infusion orifice to ensure the retraction or deployment of these corresponding features as desired. For instance as shown in FIG. 18A one of the marker bands 1722 of the guide member 1706 is shown substantially aligned with the catheter marker band 1802. Accordingly, a technician using the thrombectomy catheter 1600 could reliably recognize that the thrombectomy catheter 1600 is in the deployed position with the infusion orifice 1700 positioned outside of the aspiration lumen 1614.

Similarly, with the infusion body 1618 in the withdrawn configuration or retracted position shown in FIG. 18B the technician could use either of the marker bands 1722 relative to the catheter marker band 1802 to identify the position of the expanded member 1704 within the aspiration lumen 1614 (e.g., at the aspiration orifice 1616) to recognize the thrombectomy catheter 1600 is in the retracted position and ready for withdrawal or operation of the thrombectomy catheter to macerate a thrombus plug 1800 therein.

In still another example a marker band 1804 is provided for the expanded member 1704. In one example the marker band 1804 is used in the retracted position 18B to index the position of the expanded member or marker band 1804 relative to the catheter marker band 1802 to identify the position of the expanded member 1704 and confirm the position of the expanded member 1704 within the catheter body 1602, for instance to thereby macerate a thrombus plug 1800 or deliver or withdraw the thrombectomy catheter 1600 to or from a location of interest in the vasculature.

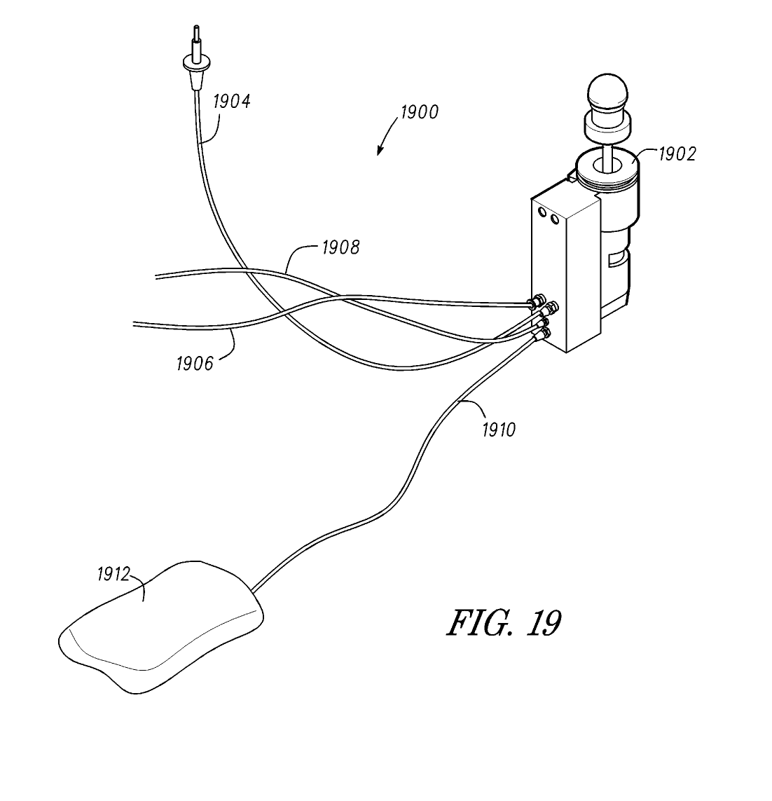

FIG. 19 shows a schematic view of a thrombectomy system 1900. The thrombectomy system 1900 is used with and includes the thrombectomy catheter 1600 previously shown in FIGS. 16, 17A, B and 18 (as well as any of the other catheter examples described herein). As shown in FIG. 19, the thrombectomy system 1900 includes an infusion pump 1902, for instance a double or single action piston based infusion pump, as well as the appropriate lines and connections to connect the thrombectomy catheter 1600 with the infusion pump, a source of infusion fluid, and an effluent bag such as the effluent bag 1912.

In one example, the infusion pump 1902 is coupled with one or more features of the thrombectomy system 1900 including an infusion source line 1904 that couples the pump a reservoir of infusion fluid such as a bag of infusion fluid (e.g., saline, lytics, medicaments or the like). In another example, the infusion pump 1902 is coupled with the thrombectomy catheter 1600 with a catheter infusion line 1906 extending from the infusion pump 1902 to the infusion fitting 1620 of the infusion body 1618. As previously shown in FIG. 16 the infusion body 1618 extends through the thrombectomy catheter 1600 to the catheter distal portion 1606 and the infusion orifice 1700 provided with the infusion body 1618.

As further shown in FIG. 19 a catheter aspiration line 1908 is provided and configured for coupling with the aspiration port 1608 of the thrombectomy catheter 1600 and the infusion pump 1902 and facilitates the delivery of effluent such as infusion fluid including entrained particulate therein through an effluent line 1910 to the effluent bag 1912. In one example the infusion pump 1902 is fitted with or coupled to an aspiration pump (as part of an overall pump assembly) configured to provide the vacuum and accordingly move the effluent through the effluent line 1910 to the effluent bag 1912.

In one example the infusion pump 1902 is a double action piston infusion pump 1902. For instance the infusion pump includes a piston as described herein configured to provide infusion fluid on both upstroke and downstroke movement of the piston within the pump body. That is to say, with translation of the piston in first and second directions infusion fluid is delivered in a substantially continuous fashion through the catheter infusion line 1906 and the catheter 1600 to the infusion orifice 1700 shown in FIG. 17 and previously described herein. In another example, the infusion pump 1902 includes a single action piston pump. In such an example the infusion pump 1902 provides infusion fluid from the infusion source line 1904 to the thrombectomy catheter 1600 and the infusion orifice 1700 as the piston is translated in a single direction, for instance on a downstroke or an upstroke depending on the pump configuration and fluid connections within the infusion pump 1902. In still other examples the thrombectomy catheter system 1900 includes an infusion pump 1902 including, but not limited to, a roller pump, a syringe injector (e.g., similar to a contract injector), or the like. Accordingly, the thrombectomy system 1900 including for instance the components shown in FIG. 19 as well as the thrombectomy catheter 1600 (and the other thrombectomy catheters described herein) is configured for use with a variety of infusion pumps 1902 including, but not limited to, the single action and double action infusion pumps described herein.

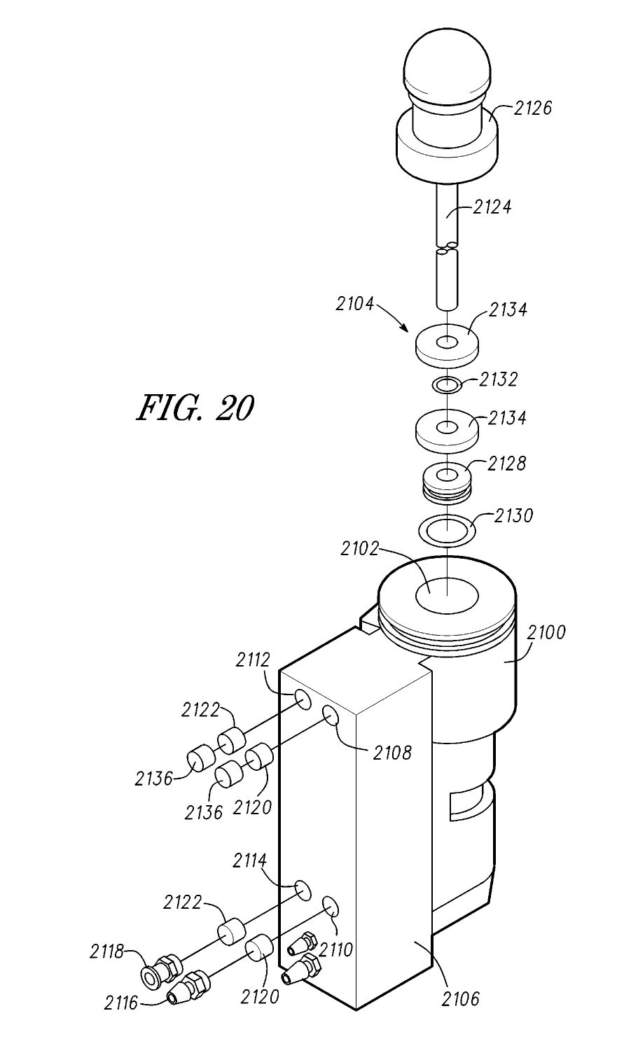

FIG. 20 shows an exploded view of the pump 1902 shown in FIG. 19. As shown in FIG. 20 this example of the 1902 is described as a double action infusion pump. As shown, the double action infusion pump 1902 includes a pump body 2100, for instance a unitary pump body formed from a single continuous piece of material. In the example, the cylinder 2102 and the pump manifold 2106 are formed as a single piece of material, for instance from a molded polymer resin. Where the double action infusion pump 1902 is constructed with a polymer, in one example the cylinder 2102 diameter and the corresponding piston 2104 diameter are enlarged to provide a high flow rate at low pressures. Accordingly, polymer fittings at the inlets and outlets, and the structural integrity of the cylinder 2102 and the piston 2104 are maintained while relatively high flow rates are realized. Optionally, the pump body 2100 is machined from aluminum, steel or the like. Accordingly, the cylinder 2102 and the corresponding inlets and outlets have increased structural integrity and the corresponding pump 1902 is operable at higher pressures and corresponding flow rates, or at higher pressures with a smaller cylinder 2102 and piston 2104.

As further shown in FIG. 20, the double action infusion pump 1902 includes a piston 2104. In one example the piston 2104 is a multicomponent piston including a series of seals configured to provide a sealing engagement between a piston disc 2128 and the cylinder 2102. The double action infusion pump 1902 includes a series of inlets and outlets in communication with first and second pump chambers formed by the movable piston 2104 and the cylinder 2102. The contemporaneous evacuation and filling of each of these pump chambers accordingly provides a continuous output of infusion fluid for instance through a manifold outlet fitting 2118 described in detail herein.

Referring again to FIG. 20, the piston 2104 is shown in an exploded configuration. In the example shown the piston 2104 includes a piston shaft 2124 having a piston fitting 2126. The piston fitting 2126 is sized and shaped for engagement with a pump motor (e.g., a pump motor configured to provide reciprocating motion to the piston 2014). The piston shaft 2124 extends to a piston seat 2134 sized and shaped to engage with a shaft seal 2132 sized and shaped to maintain a fluid seal between the piston shaft 2124 and at least the first pump chamber provided between the piston 2104 and the piston seat 2134. For instance, in one example a shaft seal 2132 is sandwiched between dual portions of the piston seat 2134 to accordingly provide a tight seal against the piston shaft 2124 and accordingly prevent the egress of fluids from the cylinder 2102. The piston shaft 2124 is slidably received within the piston seat 2134 and the shaft seal 2132 and is coupled at an opposed end to the piston disc 2128. In the example shown, the piston disc 2128 includes a piston seal 2130 sized and shaped to engage in sliding movement along the cylinder 2102. The piston 2104, including for instance the piston disc 2128 and the piston seal 2130, bifurcates the cylinder 2102 into first and second pump chambers.

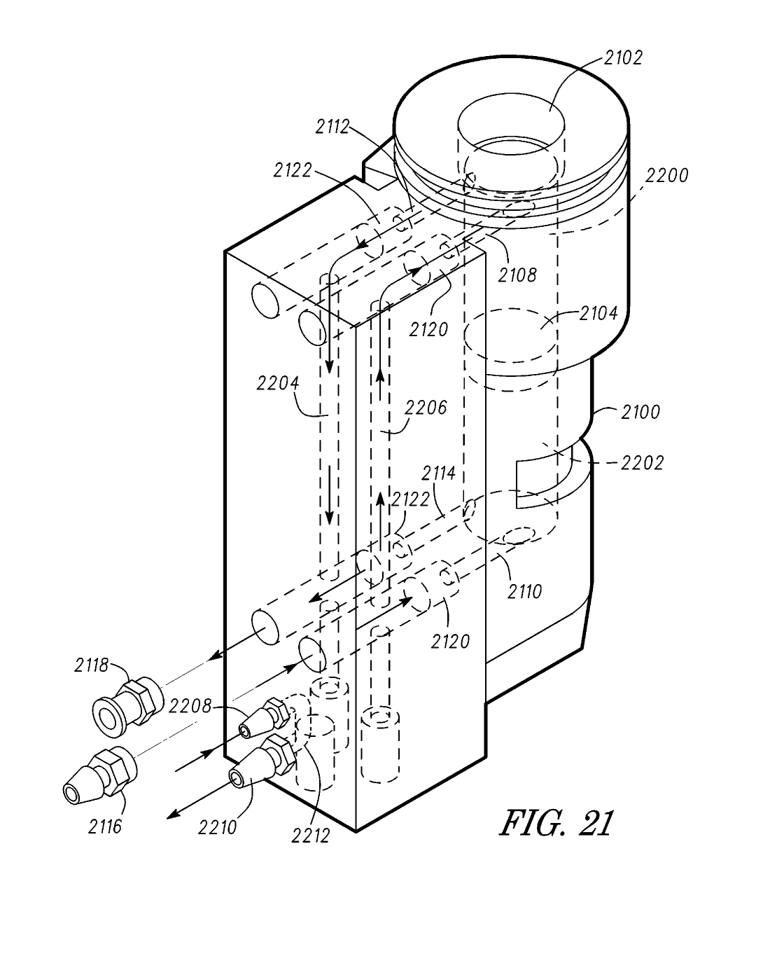

Referring again to FIG. 20 the cylinder 2102 is in communication with a first fluid inlet 2108 and a first fluid outlet 2112 extending through the pump manifold 2106. Similarly the second pump chamber (positioned relatively below the piston 2104) is in communication with a second fluid outlet 2114 and a second fluid inlet 2110. The pump manifold 2106 in another example includes a manifold inlet fitting 2116 and a manifold outlet fitting 2118. The manifold inlet fitting 2116 is optionally in communication with the first fluid inlet 2108 and the second fluid inlet 2110. As will be shown for instance in FIG. 21, the manifold inlet fitting 2116 is coupled with each of these fluid inlets 2108, 2110 to accordingly provide a source of fluid for each of the first and second pump chambers. In a similar manner, the manifold outlet fitting 2118 is in communication with the first fluid outlet 2112 and the second fluid outlet 2114 associated with the first and second pump chambers, respectively. The manifold outlet fitting 2118 is accordingly configured to couple with the catheter 1600 shown in FIG. 16 (or other exemplary catheters described herein) and provide the continuous output of fluid flow from the pump 1902 to the one or more infusion ports 1700.

As further shown in FIG. 20 the double action infusion pump 1902 includes a plurality of unidirectional valves provided in each of the inlets and outlets to accordingly ensure a unidirectional flow of fluid form each of the pump chambers. For instance, the first fluid inlet 2108 includes a unidirectional inlet valve 2120. In a similar manner, the second fluid inlet 2110 includes a unidirectional inlet valve 2120. The unidirectional inlet valves 2120 (e.g., check valves) allow the inflow of fluid for instance into the cylinder 2102 including the respective first and second pump chambers.

In a similar manner, the first and second fluid outlets 2112, 2114 correspondingly include unidirectional outlet valves 2122. The unidirectional outlet valves 2122 cooperate to ensure evacuating fluid from the cylinders 2102 is delivered out of the first fluid outlet and the second fluid outlet 2112, 2114 and is not otherwise backflowed into the cylinder 2102, for instance during reciprocation of the piston 2104 while filling of either of the first and second piston chambers. Stated another way, the unidirectional inlet valves 2120 and the unidirectional outlet valves 2122 cooperate to provide a one way flow of fluid from each of the first and second pump chambers provided within the cylinder 2102 and separated by the piston 2104. Accordingly, through reciprocation of the piston 2104 a flow of fluid is continuously provided from either of the first and second fluid outlets 2112, 2114 throughout reciprocation of the piston 204.

Optionally, the unidirectional inlet and outlet valves 2120, 2122 are reversed. In the reversed configuration the double action infusion pump 1902 is operable as a vacuum pump. For instance, in one example, the double action infusion pump 1902 or a second instance of the pump is used as an aspiration pump to accordingly draw fluid (e.g., saline and body fluids with entrained particulate) to the effluent bag 1912. Optionally, the pump in the vacuum configuration is coupled with the effluent 1912 and applies a negative pressure within the reservoir to accordingly apply suction (e.g., of one of the exemplary catheters, such as the aspiration lumen 1614 of the catheter 1600).

FIG. 21 shows another perspective view of the double action infusion pump 1902 previously shown in FIG. 20. In this view the interior of the infusion pump is provided in broken lines. For instance the cylinder 2102 is shown divided by the piston 2104 received therein. As shown in FIG. 21, the cylinder 2102 is accordingly divided into a first pump chamber 2200 and a second pump chamber 2202. The first pump chamber 2200 is in communication with the first and second fluid inlet and outlet 2108, 2112. In a similar manner, the second pump chamber 2202 is in communication with the second fluid inlet and second fluid outlet 2110, 2114. As previously described each of the first fluid inlet and second fluid inlet 2108, 2110 are in one example in communication with a manifold inlet fitting 2116. For instance, an inlet interconnect 2206 formed within the pump manifold 2106 provides communication between each of the first fluid inlet 2108 and the second fluid inlet 2110. In one example the manifold inlet fitting 2116 is in communication with a fluid source such as an infusion reservoir coupled with the pump with the infusion source line 1904 previously shown in FIG. 19.

In a similar manner to the first and second fluid inlets 2108, 2110, the first and second fluid outlets 2112, 2114 are in communication optionally with one another by way of an outlet interconnect 2204. As shown in FIG. 21 each of the outlets 2112, 2114 are in communication by way of the interconnect 2204 and accordingly provide their outputs through the manifold outlet fitting 2118, for instance to the catheter 1600. In another example, each of the first and second fluid inlets 2108, 2110 and the first and second fluid outlets 2112, 2114 are respectively interconnected directly with a catheter, such as the thrombectomy catheter 1600. For instance the pump manifold 2106 houses each of the inlets and outlets and accordingly allows for separate communication of each of the inlets and outlets with the corresponding catheter 1600 or infusion fluid source.

As further shown in FIG. 21 and as previously described herein, in one example the pump body 2100 is a unitary pump body combining one or more features into a modular component assembly configured for installation within the pump housing including a reciprocating pump motor. That is to say, the double action infusion pump 1902 including for instance a unitary pump body 2100 is loaded as a single module into the pump housing and coupled with the catheter 1600 as well as an effluent bag 1912.

In one example, the pump assembly receiving the pump 1902 includes an aspiration pump such as a roller pump, a diaphragm pump or the like interposed between the effluent bag 1912 and the 1902. The effluent pump provides a source of aspiration (e.g., a vacuum) within the thrombectomy catheter 1600 (e.g., at the aspiration orifice 1616) and accordingly moves an effluent fluid (e.g., a returning fluid from the catheter 1600 including for instance thrombus or plaque particulate therein) through the unitary pump body 2100 and thereafter into the effluent bag 1912. As shown in FIG. 21, in one example the pump body 2100 includes an aspiration inlet 2208 and an aspiration outlet 2210 formed in the pump body 2100. As further shown in FIG. 21 an aspiration passage 2212 provides communication between each of the aspiration inlet 2208 and the aspiration outlet 2210. Accordingly, the aspiration inlet and outlet 2208, 2210 cooperate to provide an effluent passage through the pump body 2100. The modular pump body 2100 installed within a pump assembly configured to receive the pump 1902 accordingly facilitates communication from the fluid source coupled with the infusion source line 1904 to the catheter 1600 and from the catheter 1600 to the effluent bag 1912.

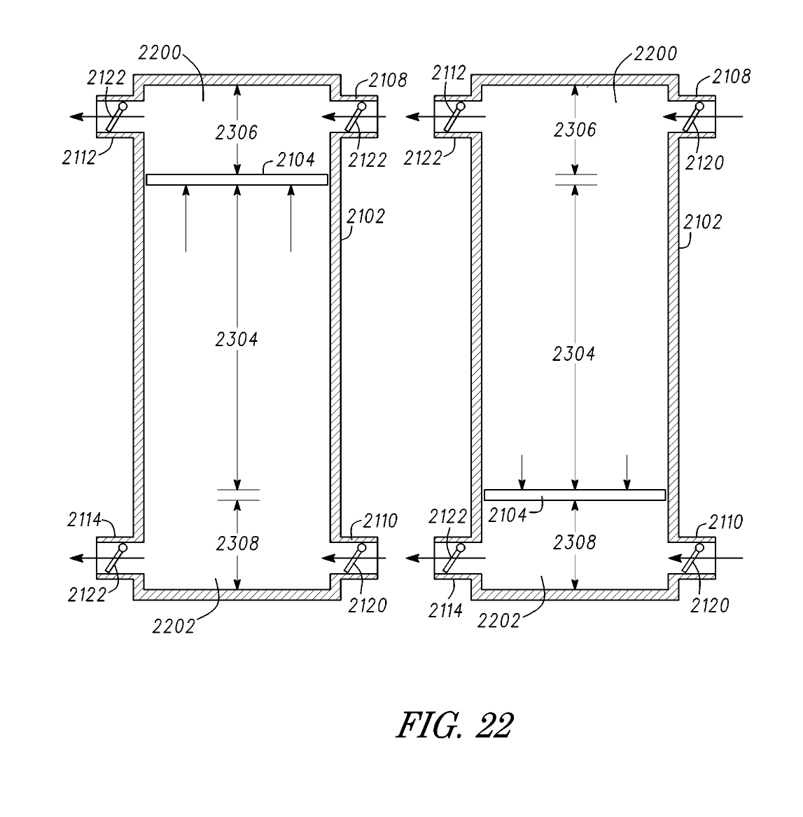

Referring now to FIG. 22 the cylinder 2102 previously shown in FIGS. 20 and 21 is shown in dual schematic representations with the piston 2104 in an upward position in the leftmost view and the piston 2104 in a lower position in the rightmost view. In both views the cylinder 2102 includes first and second pump chambers 2200, 2202 formed by the piston 2104 and the cylinder 2102. As shown between the two views the first and second pump chambers 2200, 2202 have variable volumes according to the movements of the piston 2104. The cylinder 2102 includes first and second fluid inlets 2108, 2110 and first and second fluid outlets 2112, 2114. Each of the pairs of fluid inlets and outlets are associated with one of the first and second pump chambers 2200, 2202 as shown in each of the schematic views. As further shown in the schematic views each of the inlets and outlets include corresponding unidirectional inlet valves 2120 and unidirectional outlet valves 2122 such as check valves. Check valves facilitate, in the example of the unidirectional inlet valve 2120, filling of each of the respective first and second pump chambers 2200, 2202. In contrast the unidirectional outlet valves 2122 associated with the first and second fluid outlets 2112, 2114 facilitate the evacuation of each of the first and second pump chambers 2200, 2202 for instance as the fluid within each of the chambers is pressurized during reciprocation of the piston 2104.

In operation the piston 2104 is reciprocated within the cylinder 2102 to accordingly fill and evacuate each of the first and second pump chambers 2200, 2202. For instance, in the leftmost view the piston 2104 is shown in an ascending configuration. In this configuration fluid within the first pump chamber 2200 is pressurized and delivered through the first fluid outlet 2112. In a converse manner, as the piston 2104 ascends the second pump chamber 2202 is filled for instance by a flow of fluid through the unidirectional inlet valve 2120 of the second fluid inlet 2110. Accordingly, as one of the first or second pump chambers 2200, 2202 is filling the opposed chamber is evacuating. The rightmost view of FIG. 22 shows the piston 2104 in a descending configuration. In this configuration the first pump chamber 2200 is filling for instance through the first fluid inlet 2108 while the second pump chamber 2202 is evacuating for instance by pushing pressurized fluid through the second fluid outlet 2114.

According to the views shown in FIG. 22 a near continuous flow of fluid from the double action infusion pump 1902 is provided, for instance as one of the first or second pump chambers 2200, 2202 is filling and the other is evacuating. Because one of the first and second pump chambers is evacuating during ascent or descent of the piston 2104 a substantially continuous output is provided from the double action infusion pump (excepting a momentary pause at the top and bottom of the piston 2104 travel). Similarly while one of the chambers is evacuating the other of the two chambers 2200, 2202 is filling to accordingly facilitate the continued delivery of fluid upon reciprocation of the piston 2104 in the opposed direction.

Referring again to FIG. 22 the piston 2104 is shown moving through various segments of the cylinder 2102. In one example, an intermediate segment 2304 spans a portion of the length of the cylinder 2102 between top and bottom zones 2306, 2308. The intermediate segment 2304 assumes the majority of the length of the cylinder 2102 in an example. In another example, the intermediate segment 2304 forms some portion of the cylinder 2102 less than or equal to half of the cylinder length. As shown in FIG. 22, the intermediate segment 2304 spans between positions near the inlets and outlets 2108, 2110, 2112, 2114 but is spaced from the inlets and outlets relative to the top and bottom zones 2306, 2308 that are more closely positioned relative to the respective inlets and outlets. The double action infusion pump 1902, in one example, is operated with varying speed in each of the upstroke and downstroke of the piston 2104. For instance, the piston is accelerated in top and bottom zones 2306, 2308 (during upstroke and downstroke) with an increase in infusion flow from the pump to offset the momentary pause of the piston 2014 at the top and bottom most portions of its movement (including a corresponding pause in the fluid flow rate). In the intermediate segment 2304, the piston 2014 has a near constant slower speed (around 0.01 to 2 inches per second). The varying of speed and of the piston 2014 ensures the fluid flow from the catheter (e.g., the thrombectomy catheter 1600) is substantially constant even with reciprocation pauses of the double action infusion pump 1902. Accordingly, by varying speed between the upstroke and downstroke a substantially continuous output of fluid from the pump 104 and flow of fluid at the catheter 110 are achieved.

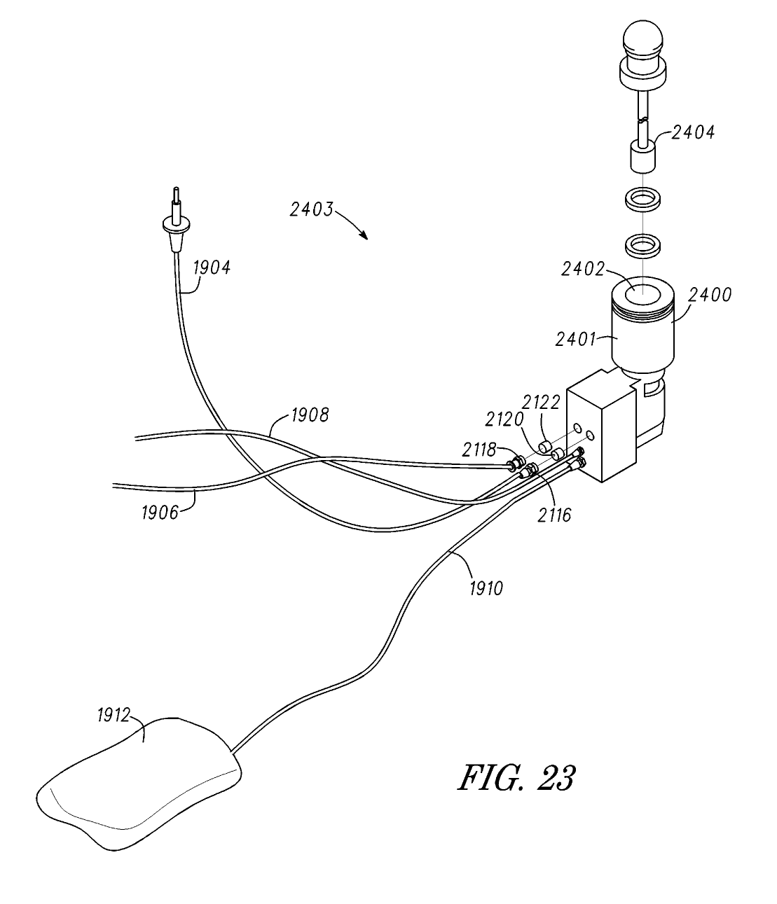

FIG. 23 shows another example of a thrombectomy system 2403 including a single action infusion pump 2400. As shown in FIG. 23, the thrombectomy system 2403 includes at least some features similar to the previously described thrombectomy system 1900. For instance, an infusion source line 1904 extends from the pump 2400 from an inlet fitting 2116 to a supply of infusion fluid (e.g., saline, lytics, medicaments or the like). A catheter infusion line 1906 extends from the pump 2400 and is configured for coupling with a catheter, for instance the thrombectomy catheter 1600. The previously described infusion fluid is directed by way of the pump 2400 through the pump body 2401 to an outlet fitting 2118 coupled with the catheter infusion line 1906. The catheter infusion line 1906 is in turn coupled with the thrombectomy catheter 1600 and thereby provides a source of infusion fluid for the thrombectomy catheter 1600 (or any of the other thrombectomy catheters previously described herein) for delivery to the infusion orifice 1700.

Additionally, the thrombectomy system 2403 includes an effluent bag 1912 coupled with the single action pump 2400 by way of an effluent line 1910 extending from a corresponding portion of the pump body 2401 optionally. The portion of the pump body is configured to allow the passage of effluent from the catheter (e.g., thrombectomy catheter 1600 previously described herein), through the catheter aspiration line 1908 to the effluent bag 1912 through the effluent line 1910. In one example, the pump body 2401 is configured for coupling within a thrombectomy assembly including one or more pumps or pump operators. For instance, a pump operator actuates the single action infusion pump 2400 and a supplemental pump (e.g., diaphragm or rolling pump) is included in the thrombectomy assembly and used as an aspiration pump configured to draw the effluent from the thrombectomy catheter 1600 through the catheter aspiration line 1908 and the effluent line 1910 to the effluent bag 1912.

In contrast to the previously described double action infusion pump 1902 shown in FIG. 19, the single action infusion pump 2400 shown in FIG. 23 provides infusion fluid on one of an upstroke or a downstroke of a piston 2404 within a cylinder 2402 of the pump body 2401. Stated another way, a single pump chamber is provided for instance beneath the piston 2404. With a piston 2404 upstroke infusion fluid is drawn from the infusion source line 1904, for instance coupled with a saline or infusion bag, through the unidirectional inlet valve 2120 and into the cylinder 2402. With the downstroke of the piston 2404, the infusion fluid within the cylinder 2402 is driven out of the cylinder 2402, for instance through the unidirectional outlet valve 2122, and through the catheter infusion line 1906 to the thrombectomy catheter 1600. Stated another way, the unidirectional inlet valve 2120 and the unidirectional outlet valve 2122 cooperate with movement of the piston 2404 (during upward and downward strokes) to draw fluid into the cylinder 2402 on an upward stroke and dispense the fluid or infuse the fluid through the catheter infusion line 1906 to the thrombectomy catheter 1600 on the downstroke of the piston 2404. In another example, the pump operation is reversed with the downstroke drawing fluid into an upper chamber within the pump body 2401 and an upstroke pushing the fluid into the thrombectomy catheter 1600.

### Additional Notes and Various Examples

Example 1 can include subject matter such as can include a thrombectomy catheter system comprising: a catheter body extending from a catheter proximal portion to a catheter distal portion, the catheter body includes: an aspiration lumen extending though the catheter body to the catheter distal portion, a catheter body inner wall extending around the aspiration lumen, and an aspiration orifice at the catheter distal portion in communication with the aspiration lumen; an infusion body including a fluid delivery lumen extending to at least one infusion orifice, the infusion body is within the aspiration lumen and extends through the aspiration orifice; and a plug removal assembly coupled with the infusion body including: an expanded member coupled to a distal end of the infusion body and located distally relative to the at least one infusion orifice, and a guide member coupled with the infusion body proximally relative to the expanded member, the guide member includes a guide outer perimeter corresponding to the catheter body inner wall, and the guide member is slidably coupled along the catheter body inner wall.

Example 2 can include, or can optionally be combined with the subject matter of Example 1, to optionally include C wherein the guide member includes a guide sleeve coupled along the infusion body.

Example 3 can include, or can optionally be combined with the subject matter of one or any combination of Examples 1 or 2 to optionally include wherein the guide sleeve is coupled with the infusion body along a sleeve wall.

Example 4 can include, or can optionally be combined with the subject matter of one or any combination of Examples 1 through 3 to optionally include wherein the guide member includes first and second sleeve rings coupled with the infusion body, and the guide sleeve wraps around the first and second sleeve rings.

Example 5 can include, or can optionally be combined with the subject matter of one or any combination of Examples 1-4 optionally to include wherein first and second marker bands wrap around the guide sleeve, and the guide sleeve is retained between the first and second sleeve rings and the first and second marker bands.

Example 6 can include, or can optionally be combined with the subject matter of Examples 1-5 to optionally include wherein the guide member is proximal relative to the at least one infusion orifice.

Example 7 can include, or can optionally be combined with the subject matter of Examples 1-6 to optionally include wherein the expanded member is slidable between deployed and plunging positions: in the deployed position the expanded member and the at least one infusion orifice are spaced from the aspiration orifice, and the guide member is within the aspiration lumen, and in the plunging position the expanded member, the at least one infusion orifice, and the guide member are within the aspiration lumen, the guide member is configured to slidably guide the expanded member into the aspiration lumen from the deployed position to the plunging position, and the expanded member is configured to plunge thrombus into the aspiration lumen.

Example 8 can include, or can optionally be combined with the subject matter of Examples 1-7 to optionally include wherein in the plunging position the expanded member closes the aspiration lumen at the catheter distal portion and isolates the infusion orifice from an exterior of the catheter body.

Example 9 can include, or can optionally be combined with the subject matter of Examples 1-8 to optionally include wherein the expanded member has a larger perimeter than the infusion body, and a corresponding perimeter to the catheter body inner wall.

Example 10 can include, or can optionally be combined with the subject matter of Examples 1-9 to optionally include a double action infusion pump configured to continuously provide a flow of infusion fluid through the fluid delivery lumen to the infusion orifice on strokes of a pump piston in first and second directions.

Example 11 can include, or can optionally be combined with the subject matter of Examples 1-10 to optionally include a single action infusion pump configured to provide a flow of infusion fluid through the fluid delivery lumen to the infusion orifice on strokes of a pump piston in a first direction.

Example 12 can include, or can optionally be combined with the subject matter of Examples 1-11 to optionally include a thrombectomy catheter system comprising: a catheter body extending from a catheter proximal portion to a catheter distal portion, the catheter body includes an aspiration lumen and an aspiration orifice at the catheter distal portion in communication with the aspiration lumen; an infusion body including at least one infusion orifice, the infusion body is within the catheter body and extends through the aspiration orifice; a plug removal assembly coupled with the infusion body including: an expanded member coupled to a distal end of the infusion body and located distally relative to the at least one infusion orifice, and a guide member coupled with the infusion body proximally relative to the expanded member, and the guide member is slidably coupled along a catheter body inner wall; an infusion pump coupled with the infusion body, the infusion pump configured to provide a flow of infusion fluid through the infusion body to the infusion orifice; and wherein the expanded member is slidable between deployed and plunging positions relative to the aspiration orifice: in the deployed position the expanded member and the at least one infusion orifice are spaced from the aspiration orifice, and in the plunging position the expanded member and the at least one infusion orifice are within the aspiration lumen, and the guide member is configured to slidably guide the expanded member into the aspiration lumen from the deployed position to the plunging position, and the expanded member is configured to plunge thrombus plugs into the aspiration lumen.

Example 13 can include, or can optionally be combined with the subject matter of Examples 1-12 to optionally include wherein the guide member includes a guide sleeve coupled along the infusion body.

Example 14 can include, or can optionally be combined with the subject matter of Examples 1-13 to optionally include wherein the guide sleeve is coupled with the infusion body along a sleeve wall.

Example 15 can include, or can optionally be combined with the subject matter of Examples 1-14 to optionally include wherein the guide member is proximal relative to the at least one infusion orifice.

Example 16 can include, or can optionally be combined with the subject matter of Examples 1-15 to optionally include wherein in the plunging position the expanded member closes the aspiration lumen at the catheter distal portion and isolates the infusion orifice from an exterior of the catheter body.

Example 17 can include, or can optionally be combined with the subject matter of Examples 1-16 to optionally include wherein the expanded member has a corresponding perimeter to a catheter body inner wall.

Example 18 can include, or can optionally be combined with the subject matter of Examples 1-17 to optionally include wherein the infusion pump includes a double action infusion pump configured to continuously provide a flow of infusion fluid through the fluid delivery lumen to the infusion orifice on strokes of a pump piston in first and second directions.

Example 19 can include, or can optionally be combined with the subject matter of Examples 1-18 to optionally include wherein the infusion pump includes a single action infusion pump configured to provide a flow of infusion fluid through the fluid delivery lumen to the infusion orifice on strokes of a pump piston in a first direction.

Example 20 can include, or can optionally be combined with the subject matter of Examples 1-19 to optionally include a method for using a thrombectomy system comprising: positioning a catheter distal portion of a catheter body adjacent to a thrombus in a vessel, the catheter body including an aspiration orifice at the catheter distal portion and an aspiration lumen in communication with the aspiration orifice; hydrodynamically removing the thrombus from the vessel with an infusion jet from at least one infusion orifice of an infusion body extending through the aspiration orifice, wherein the removed thrombus includes at least one thrombus plug larger than the aspiration orifice; and removing the at least one thrombus plug including: guiding an expanded member of the infusion body into the aspiration lumen with a guide member proximal to the expanded member on the infusion body, the guide member slidably coupled along a catheter body inner wall, plunging the at least one thrombus plug through the aspiration orifice into the aspiration lumen with the expanded member, and macerating the at least one thrombus plug within the aspiration lumen with the infusion jet from the at least one infusion orifice.

Example 21 can include, or can optionally be combined with the subject matter of Examples 1-20 to optionally include wherein hydrodynamically removing thrombus from the vessel includes: translating the infusion body, the at least one infusion orifice, and the infusion jet relative to the catheter body and the vessel, and guiding the infusion body with the guiding member during translation.

Example 22 can include, or can optionally be combined with the subject matter of Examples 1-21 to optionally include wherein guiding the expanded member includes continuously guiding the expanded member with the guide member from a deployed position for hydrodynamic removal to a plunging position for removal of the at least one thrombus plug.

Example 23 can include, or can optionally be combined with the subject matter of Examples 1-22 to optionally include wherein the guiding member includes a guide sleeve coupled with the infusion body along a sleeve wall, the guide sleeve having a corresponding perimeter to the catheter body inner wall, and guiding the expanded member of the infusion body includes guiding with the guide sleeve slidably coupled along the catheter body inner wall.

Example 24 can include, or can optionally be combined with the subject matter of Examples 1-23 to optionally include wherein plunging the at least one thrombus plug includes withdrawing the expanded member into the aspiration lumen with relative movement of the infusion body relative to the catheter body.

Example 25 can include, or can optionally be combined with the subject matter of Examples 1-24 to optionally include wherein removing the at least one thrombus plug includes closing the aspiration lumen at the catheter distal portion with the expanded member in the aspiration lumen.

Example 26 can include, or can optionally be combined with the subject matter of Examples 1-25 to optionally include wherein macerating the at least one thrombus plug includes: directing the infusion jet into the aspiration lumen with the at least one thrombus plug therein to form thrombus particulate, and directing a flow of infusion fluid with entrained thrombus particulate distally through the aspiration lumen according to the closing of the aspiration lumen.

Example 27 can include, or can optionally be combined with the subject matter of Examples 1-26 to optionally include wherein hydrodynamically removing thrombus includes at least partially plugging one or more of the aspiration orifice or the aspiration lumen with the at least one thrombus plug.

Example 28 can include, or can optionally be combined with the subject matter of Examples 1-27 to optionally include infusing fluid to the infusion body and the infusion orifice with an infusion pump.

Example 29 can include, or can optionally be combined with the subject matter of Examples 1-28 to optionally include wherein infusing fluid includes infusing with a double action infusion pump providing a continuous flow of infusion fluid on strokes of a pump piston in first and second directions.

Example 30 can include, or can optionally be combined with the subject matter of Examples 1-29 to optionally include wherein infusing fluid includes infusing with a single action infusion pump providing a flow of infusion fluid on strokes of a pump piston in a first direction.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

We claim:

1. A thrombectomy catheter system comprising:
a catheter body extending from a catheter proximal portion to a catheter distal portion, the catheter body includes:
an aspiration lumen extending though the catheter body to the catheter distal portion, a catheter body inner wall extending around the aspiration lumen, and
an aspiration orifice at the catheter distal portion in communication with the aspiration lumen;
an infusion body including a fluid delivery lumen extending to at least one infusion orifice, the infusion body is within the aspiration lumen and extends through the aspiration orifice; and
a plug removal assembly coupled with the infusion body including:
an expanded member coupled to a distal end of the infusion body and located distally relative to the at least one infusion orifice, and
a guide member coupled with the infusion body proximally relative to the expanded member, the guide member includes a guide outer perimeter corresponding to the catheter body inner wall, and the guide member is slidably coupled along the catheter body inner wall.

2. The thrombectomy catheter system of claim 1, wherein the guide member includes a guide sleeve coupled along the infusion body.

3. The thrombectomy catheter system of claim 2, wherein the guide sleeve is coupled with the infusion body along a sleeve wall.

4. The thrombectomy catheter system of claim 1, wherein the guide member includes first and second sleeve rings coupled with the infusion body, and the guide sleeve wraps around the first and second sleeve rings.

5. The thrombectomy catheter system of claim 4, wherein first and second marker bands wrap around the guide sleeve, and the guide sleeve is retained between the first and second sleeve rings and the first and second marker bands.

6. The thrombectomy catheter system of claim 1, wherein the guide member is proximal relative to the at least one infusion orifice.

7. The thrombectomy catheter system of claim 1, wherein the expanded member is slidable between deployed and plunging positions:
in the deployed position the expanded member and the at least one infusion orifice are spaced from the aspiration orifice, and the guide member is within the aspiration lumen, and
in the plunging position the expanded member, the at least one infusion orifice, and the guide member are within the aspiration lumen, the guide member is configured to slidably guide the expanded member into the aspiration lumen from the deployed position to the plunging position, and the expanded member is configured to plunge thrombus into the aspiration lumen.

8. The thrombectomy catheter system of claim 7, wherein in the plunging position the expanded member closes the aspiration lumen at the catheter distal portion and isolates the infusion orifice from an exterior of the catheter body.

9. The thrombectomy catheter system of claim 1, wherein the expanded member has a larger perimeter than the infusion body, and a corresponding perimeter to the catheter body inner wall.

10. The thrombectomy catheter system of claim 1 comprising a double action infusion pump configured to continuously provide a flow of infusion fluid through the fluid delivery lumen to the infusion orifice on strokes of a pump piston in first and second directions.

11. The thrombectomy catheter system of claim 1 comprising a single action infusion pump configured to provide a flow of infusion fluid through the fluid delivery lumen to the infusion orifice on strokes of a pump piston in a first direction.

12. A thrombectomy catheter system comprising:
a catheter body extending from a catheter proximal portion to a catheter distal portion, the catheter body includes an aspiration lumen and an aspiration orifice at the catheter distal portion in communication with the aspiration lumen;
an infusion body including at least one infusion orifice, the infusion body is within the catheter body and extends through the aspiration orifice;
a plug removal assembly coupled with the infusion body including:
an expanded member coupled to a distal end of the infusion body and located distally relative to the at least one infusion orifice, and
a guide member coupled with the infusion body proximally relative to the expanded member, and the guide member is slidably coupled along a catheter body inner wall;
an infusion pump coupled with the infusion body, the infusion pump configured to provide a flow of infusion fluid through the infusion body to the infusion orifice; and
wherein the expanded member is slidable between deployed and plunging positions relative to the aspiration orifice:
in the deployed position the expanded member and the at least one infusion orifice are spaced from the aspiration orifice, and
in the plunging position the expanded member and the at least one infusion orifice are within the aspiration lumen, and the guide member is configured to slidably guide the expanded member into the aspiration lumen from the deployed position to the plunging position, and the expanded member is configured to plunge thrombus plugs into the aspiration lumen.

13. The thrombectomy catheter system of claim 12, wherein the guide member includes a guide sleeve coupled along the infusion body.

14. The thrombectomy catheter system of claim 13, wherein the guide sleeve is coupled with the infusion body along a sleeve wall.

15. The thrombectomy catheter system of claim 12, wherein the guide member is proximal relative to the at least one infusion orifice.

16. The thrombectomy catheter system of claim 12, wherein in the plunging position the expanded member closes the aspiration lumen at the catheter distal portion and isolates the infusion orifice from an exterior of the catheter body.

17. The thrombectomy catheter system of claim 12, wherein the expanded member has a corresponding perimeter to a catheter body inner wall.

18. The thrombectomy catheter system of claim 12, wherein the infusion pump includes a double action infusion pump configured to continuously provide a flow of infusion fluid through the fluid delivery lumen to the infusion orifice on strokes of a pump piston in first and second directions.

19. The thrombectomy catheter system of claim 12, wherein the infusion pump includes a single action infusion pump configured to provide a flow of infusion fluid through the fluid delivery lumen to the infusion orifice on strokes of a pump piston in a first direction.

20. A method for using a thrombectomy catheter system comprising:
positioning a catheter distal portion of a catheter body adjacent to a thrombus in a vessel, the catheter body including an aspiration orifice at the catheter distal portion and an aspiration lumen in communication with the aspiration orifice;
hydrodynamically removing the thrombus from the vessel with an infusion jet from at least one infusion orifice of an infusion body extending through the aspiration orifice, wherein the removed thrombus includes at least one thrombus plug larger than the aspiration orifice; and
removing the at least one thrombus plug including:
guiding an expanded member of the infusion body into the aspiration lumen with a guide member proximal to the expanded member on the infusion body, the guide member slidably coupled along a catheter body inner wall,
plunging the at least one thrombus plug through the aspiration orifice into the aspiration lumen with the expanded member, and
macerating the at least one thrombus plug within the aspiration lumen with the infusion jet from the at least one infusion orifice.
